SEQUENCE LISTING (29 sequences)

SEQ ID NO: 1 - DNA, 1389 bp, Artificial Sequence
  Synthetic
atgcctaaaaagaaaaggaaggttggtgggggaggatcagctcacgtttatctgtgcaaccgcccgctgtggctgaagttccatcgtcaccagaccgagatgatcattaccaaacaaggtcgtcgtatgttcccgtttctgagcttcaacatcaacggcctgaacccgaccgcgcactacaacgtgttcgttgaagtggttctggcggacccgaaccactggcgttttcagggtggcaaatgggttacctgcggcaaggcggataacaacatgcaaggcaacaagatgtatgtgcacccggagagcccgaacaccggtagccactggatgcgtcaggaaattagctttggcaagctgaaactgaccaacaacaaaggcgcgaacaacaacaacacccagatgatcgttctgcaaagcctgcacaagtaccaaccgcgtctgcacattgtggaggttaccgaagacggcgtggaggatctgaacgaaccgagcaaaacccagaccttcacctttagcgaaacccaattcatcgcggttaccgcgtaccagaacaccgacatcacccaactgaaaattgatcacaacccgttcgcgaagggttttcgtgacaactacgatagcagccaccagattgtgccgggtggccgttatggcgttcagagcttcttcccggagccgtttgtgaacaccctgccgcaggcgcgttactataacggtgaacgtaccgttccgcaaaccaacggcctgctgagcccgcagcaaagcgaggaagtggcgaacccgccgcagcgttggctggtgaccccggttcagcaaccgggtaccaacaaactggacatcagcagctacgagagcgaatataccagcagcaccctgctgccgtacggtattaagagcctgccgctgcagaccagccatgcgctgggctactatccggatccgacctttccggcgatggcgggttggggtggccgtggcagctatcagcgtaaaatggcggcgggtctgccgtggaccagccgtaccagcccgaccgtgtttagcgaggaccaactgagcaaggaaaaagttaaagaggagatcggtagcagctggattgaaaccccgccgagcatcaaaagcctggacagcaacgatagcggcgtgtacaccagcgcgtgcaagcgtcgtcgtctgagcccgagcaacagcagcaacgagaacagcccgagcatcaagtgcgaagacattaacgcggaggaatatagcaaagataccagcaagggtatgggtggctactatgcgttttacaccaccccgggtggcggtggcagctatggccgtaagaagcgtcgtcagcgtcgtcgcggcggcggcggcagccatcaccatcaccatcacggttaatga

SEQ ID NO: 2 - protein, 461 aa, Artificial Sequence
  Synthetic
  MISC_FEATURE (3)..(8): Nuclear Localization Signal
  MISC_FEATURE (8)..(13): Glycine linker
  MISC_FEATURE (14)..(433): Eomes polypeptide
  MISC_FEATURE (433)..(438): Glycine linker
  MISC_FEATURE (439)..(449): TAT cell-penetrating peptide
  MISC_FEATURE (450)..(455): Glycine linker
  MISC_FEATURE (455)..(460): Histidine 6x Tag
MPKKKRKVGGGGSAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTPGGGGSYGRKKRRQRRRGGGGSHHHHHHG

SEQ ID NO: 3 - DNA, 2061 bp, Homo sapiens
atgcagttaggggagcagctcttggtgagctcagtgaacctgcctggcgcgcacttctacccgctggagagtgcgcgaggcggcagcggcgggagcgctggccacctccccagcgcggccccctctcctcagaagttggacttagacaaagcgtccaagaagttttccggcagtctctcctgcgaggcggtgagcggggagcccgcagccgccagcgcaggggcccccgcggccatgcttagtgacaccgacgccggggacgcatttgccagcgctgcggcagtggccaagccggggcccccggacggccgcaagggctccccctgcggggaggaggagctgccctccgccgctgcagccgccgccgccgccgccgccgcggctgcggccactgcgcgctactccatggacagcctgagctccgagcggtactacctccagtcccccggtcctcaggggtcggagctggctgcgccctgctcactcttcccgtaccaggcggcggctggggcgccccacggacctgtgtacccggctcctaacggggcgcgctacccctacggctccatgctgccccccggcggcttccccgcggctgtgtgcccacccgggagggcgcagttcggcccaggagccggtgcgggcagtggcgcgggcggtagcagcggcgggggcggcggcccgggcacctatcagtacagccagggggctccgctctacgggccgtaccctggagccgcagcggcgggatcttgcggaggactggggggcctgggggttccaggttctggcttccgtgcccacgtctacctgtgcaaccggcctctgtggctcaaattccaccgccaccaaactgagatgatcattacgaaacagggcaggcgcatgtttcctttcttgagcttcaacataaacggactcaatcccactgcccactacaatgtgttcgtagaggtggtgctggcggaccccaaccactggcgcttccaggggggcaaatgggtgacctgtggcaaagccgacaataacatgcagggcaacaaaatgtatgttcacccagagtctcctaatactggttcccactggatgagacaggagatttcattcgggaaattaaaactcaccaataacaaaggcgcaaataacaacaacacccagatgatagtcttacaatccttacacaaataccaaccccgactgcatattgttgaagttacagaggatggcgtggaggacttgaatgagccctcaaagacccagacttttaccttctcagaaacgcaattcattgcagtgactgcctaccaaaacaccgatattactcaactaaagattgatcataacccctttgcaaaaggcttcagagacaactatgattcatcccatcagattgtccctggaggtcggtacggcgttcaatccttcttcccggagccctttgtcaacactttacctcaagcccgctattataatggcgagagaaccgtgccacagaccaacggcctcctttcaccccaacagagcgaagaggtggccaaccctccccagcggtggcttgtcacgcctgtccagcaacctgggaccaacaaactagacatcagttcctatgaatctgaatatacttctagcacattgctcccatatggcattaaatccttgccccttcagacatcccatgccctggggtattacccagacccaacctttcctgcaatggcagggtggggaggtcgaggttcttaccagaggaagatggcagctggactaccatggacctccagaacaagccccactgtgttctctgaagatcagctctccaaggagaaagtgaaagaggaaattggctcttcttggatagagacacccccttccatcaaatctctagattccaatgattcaggagtatacaccagtgcttgtaagcgaaggcggctgtctcctagcaactccagtaatgaaaattcaccctccataaagtgtgaggacattaatgctgaagagtatagtaaagacacctcaaaaggcatgggagggtattatgctttttacacaactccctaa

SEQ ID NO: 4 - protein, 686 aa, Homo sapiens
MQLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTP

SEQ ID NO: 5 - DNA, 2124 bp, Mus musculus
atgcagttgggagagcagctcctggtgagctcggtgaacctgcccggcgcgcacttctactcgctggagagtgctcgcggaggaggaggaggaggaggcggaggaggagggggaggcggagggagcgtcagcctcctccccggtgctgccccctcgccccagaggctggacttagacaaagcgtccaagaagtttccgggcagtctcccgtgccaggcggggagcgcagaacccgcaggcgccggcgcgggggcccccgcggccatgctcagtgacgcggacgctggggacaccttcgggagcacctcggcggtggccaagcccgggcccccggacggccgcaagggctccccgtgcgcggaggaggagctgccctccgccgccaccgccgcggccaccgcgcgctactccatggacagcctgagctccgagcgctactacctcccgtcgccgggaccgcagggctccgagctcgccgcgccctgctcgctcttccagtacccggcggcggccggagcagcccacggacccgtgtaccccgcgtccaatggcgcgcgctacccctacggctccatgctgccccccggtggattccccgccgccgtgtgcccgcccgcgagggcgcagttcggccccgctgcgggttcggggagcggcgctggtagcagcggcggtggtgccggcggtcctggcgcctatccctacggccagggttctccgctctacgggccatacgccggaacctcagcggccgggtcttgtggaggattggggggcctaggggtgcctggctccggcttccgcgcccacgtctacctgtgcaaccggcccctatggctcaaattccaccggcaccaaactgagatgatcatcaccaaacagggcaggcgcatgtttcctttcttgagcttcaacataaacggactcaaccccaccgcccactacaatgttttcgtggaagtggttctggccgaccctaaccactggcgcttccaggggggcaagtgggtgacctgcggcaaagcggacaataacatgcagggcaataagatgtacgttcacccagaatctcctaacactggctcccactggatgaggcaggagatttcctttgggaagttaaaactcaccaataacaaaggtgcaaacaacaacaacacacagatgatagtgttgcagtctctgcacaaataccaaccgaggctgcacatcgtggaagtgacagaggacggtgtggaggacttgaatgaaccttccaagactcagaccttcaccttctcagagacacagttcatcgctgtgacggcctaccaaaacacggatatcacccagctaaagatcgaccataaccccttcgccaaaggcttccgggacaactacgattccatgtacacggcttcagaaaatgacaggttaactccatctcccacggattcccctagatcccatcagattgtccctggaggtcggtacggcgttcaaaacttcttcccggagccctttgtcaacactttgcctcaagcccgatattataacggtgagagaaccgtgccacagaccaacggcctcctctcaccccaacagagcgaagaggtggccaaccctccccagcggtggcttgtcacgcctgtccagcaacctgtgaccaacaagctagacatcggttcttatgaatctgaatatacttccagtaccttgctcccatatggtattaagtccttgcccctgcagacatcccatgccctggggtattaccctgacccgaccttccctgctatggcagggtggggaggccgtggcgcttatcagaggaagatggcagctggactaccatggacatccagaatgagcccacctgtcttcccagaagatcagcttgccaaggaaaaagttaaagaagagattagttcctcctggatagagactcccccctccatcaagtctctagactccagcgactccggggtgtacaacagcgcttgcaagagaaagcgcctgtctcccagcacccccagcaatggaaactcgccccccataaagtgtgaggacattaacactgaagagtacagtaaagacacctccaaaggcatgggggcttattatgctttttacacaagtccctag

SEQ ID NO: 6 - protein, 707 aa, Mus musculus
MQLGEQLLVSSVNLPGAHFYSLESARGGGGGGGGGGGGGGGSVSLLPGAAPSPQRLDLDKASKKFPGSLPCQAGSAEPAGAGAGAPAAMLSDADAGDTFGSTSAVAKPGPPDGRKGSPCAEEELPSAATAAATARYSMDSLSSERYYLPSPGPQGSELAAPCSLFQYPAAAGAAHGPVYPASNGARYPYGSMLPPGGFPAAVCPPARAQFGPAAGSGSGAGSSGGGAGGPGAYPYGQGSPLYGPYAGTSAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSMYTASENDRLTPSPTDSPRSHQIVPGGRYGVQNFFPEPFVNTLPQARYYNGERTVPQTNGLLSPQQSEEVANPPQRWLVTPVQQPVTNKLDIGSYESEYTSSTLLPYGIKSLPLQTSHALGYYPDPTFPAMAGWGGRGAYQRKMAAGLPWTSRMSPPVFPEDQLAKEKVKEEISSSWIETPPSIKSLDSSDSGVYNSACKRKRLSPSTPSNGNSPPIKCEDINTEEYSKDTSKGMGAYYAFYTSP

SEQ ID NO: 7 - protein, 21 aa, Artificial Sequence
  Synthetic
KETWWETWWTEWSQPKKKRKV

SEQ ID NO: 8 - protein, 20 aa, Artificial Sequence
  Synthetic
AVKRPAATKKAGQAKKKKLD

SEQ ID NO: 9 - protein, 25 aa, Artificial Sequence
  Synthetic
MSRRRKANPTKLSENAKKLAKEVEN

SEQ ID NO: 10 - protein, 9 aa, Artificial Sequence
  Synthetic
PAAKRVKLD

SEQ ID NO: 11 - protein, 9 aa, Artificial Sequence
  Synthetic
KLKIKRPVK

SEQ ID NO: 12 - protein, 4 aa, Artificial Sequence
  Synthetic
  MISC_FEATURE (2)..(2): X is K or R
  MISC_FEATURE (3)..(3): X is any amino acid
  MISC_FEATURE (4)..(4): X is K or R
KXXX

SEQ ID NO: 13 - protein, 14 aa, Artificial Sequence
  Synthetic
YGRKKRRQRRRPPQ

SEQ ID NO: 14 - protein, 16 aa, Artificial Sequence
  Synthetic
RQIKIWFQNRRMKWKK

SEQ ID NO: 15 - protein, 26 aa, Artificial Sequence
  Synthetic
GWTLNSAGYLLGKINLKALAALAKKL

SEQ ID NO: 16 - protein, 7 aa, Artificial Sequence
  Synthetic
LCLRPVG

SEQ ID NO: 17 - protein, 10 aa, Artificial Sequence
  Synthetic
  MISC_FEATURE (9)..(10): Optional; length can be 8, 9, or 10 residues
RRRRRRRRRR

SEQ ID NO: 18 - protein, 10 aa, Artificial Sequence
  Synthetic
  MISC_FEATURE (9)..(10): Optional; length can be 8, 9, or 10 residues
KKKKKKKKKK

SEQ ID NO: 19 - protein, 18 aa, Artificial Sequence
  Synthetic
KLALKLALKALKAALKLA

SEQ ID NO: 20 - protein, 21 aa, Artificial Sequence
  Synthetic
KETWWETWWTEWSQPKKKRKV

SEQ ID NO: 21 - protein, 12 aa, Artificial Sequence
  Synthetic
PLILLRLLRGQF

SEQ ID NO: 22 - protein, 12 aa, Artificial Sequence
  Synthetic
PLIYLRLLRGQF

SEQ ID NO: 23 - protein, 15 aa, Artificial Sequence
  Synthetic
KLWMRWYSPTTRRYG

SEQ ID NO: 24 - protein, 18 aa, Artificial Sequence
  Synthetic
KLALKLALKALKAALKLA

SEQ ID NO: 25 - protein, 18 aa, Artificial Sequence
  Synthetic
LLIILRRRIRKQAHAHSK

SEQ ID NO: 26 - protein, 10 aa, Artificial Sequence
  Synthetic
RRIPNRRPRR

SEQ ID NO: 27 - protein, 12 aa, Artificial Sequence
  Synthetic
RRQRRTSKLMKR

SEQ ID NO: 28 - protein, 5 aa, Artificial Sequence
  Synthetic
GGGGS

SEQ ID NO: 29 - DNA, 6642 bp, Artificial Sequence
  Synthetic
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgcctaaaaagaaaaggaaggttggtgggggaggatcagctcacgtttatctgtgcaaccgcccgctgtggctgaagttccatcgtcaccagaccgagatgatcattaccaaacaaggtcgtcgtatgttcccgtttctgagcttcaacatcaacggcctgaacccgaccgcgcactacaacgtgttcgttgaagtggttctggcggacccgaaccactggcgttttcagggtggcaaatgggttacctgcggcaaggcggataacaacatgcaaggcaacaagatgtatgtgcacccggagagcccgaacaccggtagccactggatgcgtcaggaaattagctttggcaagctgaaactgaccaacaacaaaggcgcgaacaacaacaacacccagatgatcgttctgcaaagcctgcacaagtaccaaccgcgtctgcacattgtggaggttaccgaagacggcgtggaggatctgaacgaaccgagcaaaacccagaccttcacctttagcgaaacccaattcatcgcggttaccgcgtaccagaacaccgacatcacccaactgaaaattgatcacaacccgttcgcgaagggttttcgtgacaactacgatagcagccaccagattgtgccgggtggccgttatggcgttcagagcttcttcccggagccgtttgtgaacaccctgccgcaggcgcgttactataacggtgaacgtaccgttccgcaaaccaacggcctgctgagcccgcagcaaagcgaggaagtggcgaacccgccgcagcgttggctggtgaccccggttcagcaaccgggtaccaacaaactggacatcagcagctacgagagcgaatataccagcagcaccctgctgccgtacggtattaagagcctgccgctgcagaccagccatgcgctgggctactatccggatccgacctttccggcgatggcgggttggggtggccgtggcagctatcagcgtaaaatggcggcgggtctgccgtggaccagccgtaccagcccgaccgtgtttagcgaggaccaactgagcaaggaaaaagttaaagaggagatcggtagcagctggattgaaaccccgccgagcatcaaaagcctggacagcaacgatagcggcgtgtacaccagcgcgtgcaagcgtcgtcgtctgagcccgagcaacagcagcaacgagaacagcccgagcatcaagtgcgaagacattaacgcggaggaatatagcaaagataccagcaagggtatgggtggctactatgcgttttacaccaccccgggtggcggtggcagctatggccgtaagaagcgtcgtcagcgtcgtcgcggcggcggcggcagccatcaccatcaccatcacggttaatgaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

(19) United States
(12) Patent Application Publication
(10) Pub. No.: US 2024/0301019 A1
(43) Pub. Date: Sep. 12, 2024

(54) RECOMBINANT EOMES RESTORES ANTI-CANCER ACTIVITY OF IMMUNE CELLS

(71) Applicant: ARIZONA BOARD OF REGENTS ON BEHALF OF THE UNIVERSITY OF ARIZONA, Tucson, AZ (US)

(72) Inventors: Young-Wook WON, Tucson, AZ (US); Seungmin HAN, Tucson, AZ (US); David A. BULL, Tucson, AZ (US)

(21) Appl. No.: 18/262,648

(22) PCT Filed: Feb. 14, 2022
(86) PCT No.: PCT/US2022/016260

§ 371 Date: Jul. 24, 2023

Related U.S. Application Data
(60) Provisional application No. 63/149,417, filed on Feb. 15, 2021.

(51) Int. Cl. C07K 14/47; A61K 35/17; A61K 38/00; A61P 35/00; C12N 5/0783

(52) U.S. Cl. CPC C07K 14/4705; A61K 35/17; A61P 35/00; C12N 5/0646; A61K 38/00; C07K 2319/09; C07K 2319/10; C07K 2319/21

(57) ABSTRACT

Described herein is a recombinant Eomes protein that restores the cytotoxic activity of exhausted immune cells. This protein comprises, a nuclear localization sequence (NLS), the transcription factor associated domain of Eomesodermin (Eomes), and a protein-transduction domain (PTD). The NLS-Eomes-PTD polypeptide spontaneously internalizes into NK cells and travels to the nucleus to control the transcription of its down-stream signaling pathways. Introduction of the NLS-Eomes-PTD polypeptide into ExNK cells (i) decreases the expression of an inhibitory antigen on ExNK cells; (ii) increases cytolytic activity: (iii) enhances cytokine secretion; (iv) improves proliferation; and (v) inhibits tumor growth.

### CROSS REFERENCE TO RELATED APPLICATIONS

This application claims priority to U.S. Provisional Patent Application No. 63/149,417, filed on Feb. 15, 2021, the contents of which are incorporated by reference herein in its entirety.

### FEDERALLY SPONSORED RESEARCH

This invention was made with government support under grant number HL138242 awarded by the National Institutes of Health. The government has certain rights in the invention.

### REFERENCE TO SEQUENCE LISTING

This application is filed with a Computer Readable Form of a Sequence Listing in accord with 37 C.F.R. § 1.821(c). The text file submitted by EFS, “212443-9012-WO01_sequence_listing_14 Feb. 2022_ST25.txt,” was created on Feb. 14, 2022, contains 30 sequences, has a file size of 37.7 Kbytes, and is hereby incorporated by reference in its entirety.

### TECHNICAL FIELD

Described herein is a recombinant Eomes protein that restores the cytotoxic activity of exhausted immune cells. This protein comprises, a nuclear localization sequence (NLS), the transcription factor associated domain of Eomesodermin (Eomes), and a protein-transduction domain (PTD). The NLS-Eomes-PTD polypeptide spontaneously internalizes into NK cells and travels to the nucleus to control the transcription of its down-stream signaling pathways. Introduction of the NLS-Eomes-PTD polypeptide into ExNK cells (i) decreases the expression of an inhibitory antigen on ExNK cells; (ii) increases cytolytic activity; (iii) enhances cytokine secretion; (iv) improves proliferation; and (v) inhibits tumor growth.

### BACKGROUND

Immune cell-based therapies have shown promise to treat various cancers. Those therapies utilize T cells, Natural Killer (NK) cells, cytokine-induced killer (CIK) cells, dendritic cells (DC), or peripheral blood mononuclear cells (PBMC). To improve the therapeutic efficacy, such immune cells are engineered or expanded ex vivo. In many cases, immune cells become “exhausted” and these exhausted immune cells are not as effective as healthy cells in the eradication of cancer cells and the treatment of cancer patients. For example, NK cells, components of innate immunity, loose their activity through senescence, chronic activation, or chemotherapy. This dysfunctional state is termed “exhausted.” Immune cell exhaustion causes the cells to lose their functionality and reduces cytotoxicity and cytokine secretion. Exhausted NK (ExNK) cells have poor cytotoxicity and cytokine secretion and are ineffective for treating cancer. Various attempts including immune checkpoint inhibitors, pre-conditioning with activating cytokines, or genetic engineering have been tried to revive exhausted ExNK cells. These technologies only restore the anti-cancer activity of ExNK cells in the short-term but are not able to rejuvenate ExNKs for long periods of time. ExNK cells treated with immune checkpoint inhibitors or activating cytokines exhibit improved efficacy only shortly after treatment. CAR technology up-regulates anti-cancer signaling pathways in an uncontrolled manner and exacerbates the exhausted state.

What is needed are compositions and methods for rejuvenating exhausted natural killer cells.

### SUMMARY

One embodiment described herein is a nucleotide sequence encoding a polypeptide, wherein the polypeptide comprises an Eomesodermin domain, and one or more nuclear localization signals and/or protein-transduction domain sequences. In one aspect, the Eomesodermin domain comprises an Eomesodermin transcription factor associated domain. In another aspect, the polypeptide comprises one or more nuclear localization signals, the Eomesodermin transcription factor associated domain, and one or more protein-transduction domains, each optionally separated by a glycine-serine linker. In one aspect, the polypeptide has the structure: NLS-GL-Eomes-TFAD-GL-PTD; PTD-GL-Eomes-TFAD-GL-NLS; PTD-GL-Eomes-TFAD; or NLS-GL-Eomes-TFAD; wherein NLS is a nuclear localization signal; GL is an optional glycine-serine linker; Eomes-TAD is an Eomesodermin transcription factor associated domain, PTD is a protein-transduction domain. In another aspect, the polypeptide further comprises an affinity tag. In another aspect, the affinity tag comprises a 6×-histidine tag. In another aspect, the Eomesodermin transcription factor associated domain comprises SEQ ID NO: 2 or 4. In another aspect, the Eomesodermin transcription factor associated domain comprises residues 267-686 of SEQ ID NO: 2 or 4. In another aspect, the nuclear localization signal comprises one or more of SEQ ID NO: 7-11 and the protein-transduction domain comprises one or more of SEQ ID NO: 13-27. In another aspect, the nuclear localization signal comprises residues 16-21 of SEQ ID NO: 7; the protein-transduction domain comprises residues 1-11 of SEQ ID NO: 13; and the glycine-serine linker comprises SEQ ID NO: 28. In another aspect, the nucleotide sequence has 85% to 99% identity to SEQ ID NO: 1. In another aspect, the nucleotide sequence is SEQ ID NO: 1.

Another embodiment described herein is a polynucleotide vector comprising a nucleotide sequence described herein.

Another embodiment described herein is a cell comprising one or more nucleotide sequences of described herein or a polynucleotide described herein.

Another embodiment described herein is a polypeptide encoded by the nucleotide sequence described herein.

Another embodiment described herein is a polypeptide encoded by the nucleotide sequence described herein, wherein the polypeptide has 85% to 99% identity to SEQ ID NO: 2.

A polypeptide encoded by the nucleotide sequence described herein, wherein the polypeptide is SEQ ID NO: 2.

Another embodiment described herein is a research tool comprising a polypeptide encoded by the nucleotide sequence described herein.

Another embodiment described herein is a therapeutic reagent comprising a polypeptide encoded by the nucleotide sequence described herein.

Another embodiment described herein is a means or method for manufacturing the nucleotide sequence described herein or a polypeptide encoded by the nucleotide sequence described herein, the process comprising: transforming or transfecting a cell with a nucleic acid as described herein; growing the cells; optionally, harvesting the cells and isolating quantities of the nucleotide sequence described herein; inducing expression of a polypeptide encoded by the nucleotide sequence described herein; harvesting the cells; and isolating and purifying the polypeptide.

Another embodiment is a nucleotide sequence described herein or a polypeptide encoded by the nucleotide sequence described herein, each produced by the means or methods described herein.

Another embodiment described herein is a therapeutic polypeptide comprising the amino acid sequence of SEQ ID NO: 2.

Another embodiment described herein is a method for rejuvenating exhausted natural killer cells, the method comprising: obtaining exhausted natural killer cells (ExNK); contacting the natural killer cells with a quantity of a polypeptide comprising one or more nuclear localization signals, an Eomesodermin transcription factor associated domain, and one or more protein-transduction domains; incubating the ExNK and polypeptide for a period of time sufficient for the polypeptide to be uptaken by the cells (transduced); and allowing the cells to incubate for a period of time sufficient for the polypeptide to rejuvenate the ExNKs.

Another embodiment described herein is a method for treating cancer, the method comprising: (a) administering a therapeutically effective amount of polypeptide comprising one or more nuclear localization signals, an Eomesodermin transcription factor associated domain, and one or more protein-transduction domains to a subject in need thereof; or (b) contacting autologous exhausted natural killer cells isolated from a subject with a therapeutically effective amount of polypeptide comprising one or more nuclear localization signals, an Eomesodermin transcription factor associated domain, and one or more protein-transduction domains; permitting the cells to rejuvenate; and administering the cells to a subject in need thereof;

Another embodiment described herein is the use of a polypeptide comprising one or more nuclear localization signals, an Eomesodermin transcription factor associated domain, and one or more protein-transduction domains for the rejuvenation of exhausted natural killer cells or the treatment of a subject in need thereof.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a cartoon showing exhaustion of effector Natural Killer (NK) cells and reactivation. FIG. 1B is a cartoon showing reactivation of NK cells by recombinant Eomes.

FIG. 2A shows an SDS gel image of Eomes expression in E. coli. FIG. 2B shows SDS and western blot images confirming expression of the recombinant Eomes protein.

FIG. 3 shows fluorescent images of Eomes transduction on NK cells. DAPI: blue and FITC: green.

FIG. 4 shows flow cytometry data of non-treated control (grey) and Eomes-NK (red).

FIG. 5 shows the optimization of transduction time. CD122 expression level are increased due to Eomes transduction. TD: transduction

FIG. 6 shows the optimization of priming time. CD122 expression of Eomes-NK cells was increased at around 24 h priming time after transduction.

FIG. 7 shows NKG2A expression in NK and ExNK cells. Red histogram: NK cells, blue histogram: ExNK cells.

FIG. 8A-C show the anti-cancer efficacy and cytokine secretion (IFN-γ, Granzyme B, TNF-α, GM-CSF) of ExNK and Eomes-ExNK cells in various cancer cell types, including Calu3 (FIG. 8A), MDA-MB-231 (FIG. 8B), and MCF7 (FIG. 8C) cancer cell lines.

FIG. 9A-B show the anti-cancer efficacy of NK, Eomes-NK, ExNK, and Eomes-ExNK cells on MDA-MB-231 breast cancer cells with various E:T ratios (FIG. 9A). NK refers to fresh NK, and ExNK refers to exhausted NK. FIG. 9B shows two graphs with merged data of untreated- and Eomes-treated cells of NK and ExNK.

FIG. 10A-B show cytokine secretion (IFN-γ, Granzyme B, TNF-α, GM-CSF) of NK and Eomes-NK cells (FIG. 10A), and ExNK and Eomes-ExNK cells (FIG. 10B) in MDA-MB-231 breast cancer cells with various E:T ratios.

FIG. 11 shows the Eomes-mediated anti-cancer effects in NK cells with various exhaustion stages. Cell death refers to cancer cell death. The black bar represents spontaneous cancer cell death.

FIG. 12A-C show Eomes-mediated cytokine secretion changes (IFN-γ, TNF-α, Granzyme B, GM-CSF) on NK cells with various exhaustion stages, including fresh NK (FIG. 12A), Mid ExNK (FIG. 12B), and ExNK (FIG. 12C).

FIG. 13A-C show the expression of the inhibitory markers NKG2A (FIG. 13A), TIM3 (FIG. 13B), and KIR (FIG. 13C) in ExNK, Eomes-ExNK, Eomes-ExNK 10 days post-transduction (D+10), and NK cells. The red and black numbers above the plots indicate the population of the number 1 and 2 plot boxes, respectively, where the black colored number represents the population of high expression level of the inhibitory marker. The bar graphs represent the population percentage that expresses antigen (black colored number on dot plots). ExNK is set at 100% as a standard.

FIG. 14A-B show expression changes of the activation antigens NKG2D (FIG. 14A) and NKp46 (FIG. 14B) in NK, Eomes-NK, ExNK, and Eomes-ExNK. The black and red numbers above the plots indicate the population of the number 1 and 2 plot boxes, respectively, where the red colored number represents the population of high expression level of the activation marker. The bar graphs represent the population percentage that expresses antigen (red colored number on dot plots). NK is set at 100% as a standard. Eomes was transduced as described herein (3 h transduction, 24 h priming). These data indicate that Eomes induces an increase in the expression of the activation markers on both NK and ExNK cells, which is in line with Eomes-mediated activated cytotoxicity.

FIG. 15 shows the improved proliferation of ExNK and NK cells upon treatment with Eomes. The graph shows proliferation of NK, Eomes-NK, ExNK, and Eomes-ExNK. These data demonstrate that Eomes protein increases the proliferation of both NK and ExNK, and that ExNK had slightly lower proliferation activity compared to NK cells. The data show a prolonged effect until about 21 days. Eomes was transduced as described herein (3 h transduction, 24 h priming).

FIG. 16A-D show in vivo anti-cancer efficacy of NK, Eomes-NK, ExNK and Eomes-ExNK cells compared to control (CTRL). FIG. 16A shows the change in average tumor volume over time. FIG. 16B shows the change in relative body weight over time. FIG. 16C shows the probability of survival over time. FIG. 16D shows the change in individual tumor volumes over time. No changes in body weight were observed after NK or ExNK cells were injected.

FIG. 17A-B show up-regulation of death receptor ligands of NK cells through Eomes. Expression changes of activation antigen in NK, Eomes-NK. ExNK, and Eomes-ExNK for FasL (FIG. 17A) and TRAIL (FIG. 17B) are shown. The black and red numbers above the plots indicate the population of the number 1 and 2 plot boxes, respectively, where the red colored number represents the population of high expression level of the activation marker. The bar graphs in FIG. 17A-B represent the red colored number, where the population percentage of NK cells is set at 100% as a standard. Eomes was transduced as described herein (3 h transduction, 24 h priming). The data indicate that Eomes induces an increase of death receptor ligands on both NK and ExNK cells, which is in line with Eomes-mediated activated cytotoxicity.

FIG. 18A-B show induction and up-regulation of the ADCC marker CD16 of NK cells through Eomes. NK-92 cells do not naturally express CD16. However, Eomes were found to induce CD16 expression on both NK ((FIG. 18A) and ExNK (FIG. 18A) cells. The black and red numbers above the plots indicate the population of the number 1 and 2 plot boxes, respectively, where the red colored number represents the population of high expression level of the activation marker. Eomes was transduced as described herein (3 h transduction, 24 h priming). The data indicate that Eomes induces an increase of CD16 activation marker on both NK and ExNK cells, which is in line with Eomes-mediated activated cytotoxicity.

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present document, including definitions, will control. Preferred methods and materials are described below, although methods and materials similar or equivalent to those described herein can be used in practice or testing of the present invention.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W. or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to #10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, the term “cancer” includes, but is not limited to, the following cancers: cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's lymphoma, non-Hodgkins lymphoma, hairy cells, leukemia, or combinations thereof.

Described herein is a recombinant Eomes protein that restores the cytotoxic activity of exhausted immune cells. This protein comprises, a nuclear localization sequence (NLS), the transcription factor associated domain of Eomesodermin (Eomes), and a protein-transduction domain (PTD). The protein transduction domain (PTD) is also known as a cell-penetrating peptide domain (CPP). The NLS-Eomes-PTD polypeptide spontaneously internalizes into NK cells and travels to the nucleus to control the transcription of its down-stream signaling pathways. Introduction of the NLS-Eomes-PTD polypeptide into ExNK cells (i) decreases the expression of an inhibitory antigen on ExNK cells; (ii) increases cytolytic activity; (iii) enhances cytokine secretion; (iv) improves proliferation; and (v) inhibits tumor growth.

Described herein is a recombinant Eomes protein that restores the cytotoxic activity of exhausted immune cells. This protein comprises, a nuclear localization sequence (NLS), the transcription factor associated domain of Eomesodermin (Eomes), and a protein-transduction domain (PTD) (also called a cell-penetrating peptide domain (CPP)), e.g., “NLS-Eomes-PTD.” Previous studies showed that Eomes is a critical transcription factor to restore the anti-tumor activity of NK cells. See Gill et al., Blood 119(24): 575805768 (2012). Eomes is down-regulated in exhausted natural killer cells as compared to functional NK cells. The NLS-Eomes-PTD polypeptide spontaneously internalizes into NK cells and travels to the nucleus to control the transcription of its down-stream signaling pathways. Introduction of the NLS-Eomes-PTD polypeptide into ExNK cells (i) decreases the expression of an inhibitory antigen on ExNK cells; (ii) increases cytolytic activity; (iii) enhances cytokine secretion; and (iv) inhibits tumor growth.

With restoring the activity of ExNK cells, the efficacy of NK cell therapies is improved and can be predictable. Also, the rejuvenated NK cells can be further modified with any technology currently available including CAR technology. To develop a new efficient method to rejuvenate ExNK cells for a prolonged period of time, the molecular target inside NK cells has to be identified in the upstream signaling pathway such includes a transcriptional factor. Eomesodermin (Eomes) and T-bet are known to regulate the maturation of NK cell. Eomes and T-bet are found down-regulated in ExNK cells. Gill et al. reported that Eomes is more critical than T-bet in the recovery of the exhausted state of NK cells. Gil et al. Blood 119(24): 575805768 (2012). The transcriptional Eomes protein has a positive feedback loop with CD122, interleukin-2 receptor beta, both of which bind with IL-2 and IL-15 for maintaining survival, cytolytic activity, and homeostasis of NK cells. Moreover, Eomes directly controls the activation of promoters of interferon-gamma, granzyme B, and perforin, all of which are cytokines associated with cancer cell killing. A recombinant fusion protein was constructed composed of Eomes, TAT, and Nucleus localization signal (NLS), which is capable of spontaneous internalization into NK cells followed by internalization into nucleus. This is because, as Eomes regulates transcription of NK cells, the final destination of Eomes in NK cells is the nucleus. TAT, a well-known protein transduction domain, allows Eomes protein penetrate the cell membrane and NLS then enables the migration of Eomes into the nucleus. Based on the finding that Eomes regulates CD122 and cytolytic cytokines, the recombinant Eomes fusion proteins described herein can recover the homeostasis and the anti-cancer activity of ExNK cells.

The NLS-Eomes-PTD polypeptides can be used as stand-alone therapeutics, adjuvants to improve the acitivity of immune cells being used in adoptive immune cell transfer; or treated NK cells can be used as an activity-enhanced NK cell therapy. The protein can be added to autologous ExNK cells at the end of ex vivo expansion prior to infusion into a patient or the treated allogeneic ExNK cells can be cryopreserved for future use. Such treatments are applicable to adoptive NK cell transfer therapy for elder-, obese-, chronic inflammed-patient, patients with prior chemotherapy, or patients who have exhausted NK cells or poorly active NK cells. Beyond NK cells, this protein is applicable to other innate immune cells or adaptive immune cells and the protein-treated NK cell therapy is not restricted to the treatment of cancer.

In one embodiment, the transcription factor associated domain of Eomesodermin (Eomes) comprises residues 267-686 of SEQ ID NO: 4 or residues 269-707 of SEQ ID NO: 6. Other transcription factor associated domain of Eomesodermin proteins from other organisms may be useful for the construction of nucleotide or polypeptide constructs as described herein.

TABLE 1
Eomesodermin Polypeptides

Human Eomesodermin Homolog Isoform 2 (SEQ ID NO: 4; encoded by SEQ ID NO: 3)
NCBI Reference Sequence: NP_005433.2
MQLGEQLLVSSVNLPGAHFYPLESARGGSGGSAGHLPSAAPSPQKLDLDKASKKFSGSLSCEAVSGEPAAASAGAP
AAMLSDTDAGDAFASAAAVAKPGPPDGRKGSPCGEEELPSAAAAAAAAAAAAAATARYSMDSLSSERYYLQSPGPQ
GSELAAPCSLFPYQAAAGAPHGPVYPAPNGARYPYGSMLPPGGFPAAVCPPGRAQFGPGAGAGSGAGGSSGGGGGP
GTYQYSQGAPLYGPYPGAAAAGSCGGLGGLGVPGSGFRAHVYLCNRPLWLKFHRHQTEMIITKQGRRMFPFLSFNI
NGLNPTAHYNVFVEVVLADPNHWRFQGGKWVTCGKADNNMQGNKMYVHPESPNTGSHWMRQEISFGKLKLTNNKGA
NNNNTQMIVLQSLHKYQPRLHIVEVTEDGVEDLNEPSKTQTFTFSETQFIAVTAYQNTDITQLKIDHNPFAKGFRD
NYDSSHQIVPGGRYGVQSFFPEPFVNTLPQARYYNGERTVPQTNGLLSPQQSEEVANPPQRWLVTPVQQPGTNKLD
ISSYESEYTSSTLLPYGIKSLPLQTSHALGYYPDPTFPAMAGWGGRGSYQRKMAAGLPWTSRTSPTVFSEDQLSKE
KVKEEIGSSWIETPPSIKSLDSNDSGVYTSACKRRRLSPSNSSNENSPSIKCEDINAEEYSKDTSKGMGGYYAFYT
TP
Mouse Eomesodermin Homolog Isoform 1 (SEQ ID NO: 6; encoded by SEQ ID NO: 5)
NCBI Reference Sequence: NP_034266
MQLGEQLLVSSVNLPGAHFYSLESARGGGGGGGGGGGGGGGSVSLLPGAAPSPQRLDLDKASKKFPGSLPCQAGSA
EPAGAGAGAPAAMLSDADAGDTFGSTSAVAKPGPPDGRKGSPCAEEELPSAATAAATARYSMDSLSSERYYLPSPG
PQGSELAAPCSLFQYPAAAGAAHGPVYPASNGARYPYGSMLPPGGFPAAVCPPARAQFGPAAGSGSGAGSSGGGAG
GPGAYPYGQGSPLYGPYAGTSAAGSCGGLGGLGVPGSGFRAHVYLCNRPLWLKFHRHQTEMIITKQGRRMFPFLSF
NINGLNPTAHYNVEVEVVLADPNHWRFQGGKWVTCGKADNNMQGNKMYVHPESPNTGSHWMRQEISFGKLKLTNNK
GANNNNTQMIVLQSLHKYQPRLHIVEVTEDGVEDLNEPSKTQTFTFSETQFIAVTAYQNTDITQLKIDHNPFAKGF
RDNYDSMYTASENDRLTPSPTDSPRSHQIVPGGRYGVQNFFPEPFVNTLPQARYYNGERTVPQINGLLSPQQSEEV
ANPPQRWLVTPVQQPVTNKLDIGSYESEYTSSTLLPYGIKSLPLQTSHALGYYPDPTFPAMAGWGGRGAYQRKMAA
GLPWTSRMSPPVFPEDQLAKEKVKEEISSSWIETPPSIKSLDSSDSGVYNSACKRKRLSPSTPSNGNSPPIKCEDI
NTEEYSKDTSKGMGAYYAFYTSP
Underlining indicates the transcription factor associated region of the polypeptide. For human Eomes this is residues 267-686 of SEQ ID NO: 2. For mouse this is residues 269-707 of SEQ ID NO: 6. Homologous residues in other Eomesodermin proteins are believed to have similar transcriptional activity.

Various nucleal localization sequences and protein-transduction domain sequences can be used to create the NLS-Eomes-PTD polypeptides described herein. Specific nuclear localization signal peptides can comprise all or specific portions of SEQ ID NO: 7-11. Likewise, specific protein-transduction domains can comprise all or specific portions of SEQ ID NO: 13-27.

TABLE 2
Protein-Transduction Domain (PTD) and Nuclear Localization Signal (NLS)
Sequences
Nuclear Localization Signal Sequences

| Name | Sequence | SEQ ID NO: |
|---|---|---|
| SV40 Large T-antigen | KETWWETWWTEWSQPKKKRKV | SEQ ID NO: 7 |
| Nucleoplasmin | AVKRPAATKKAGQAKKKKLD | SEQ ID NO: 8 |
| EGL-13 | MSRRRKANPTKLSENAKKLAKEVEN | SEQ ID NO: 9 |
| c-Myc | PAAKRVKLD | SEQ ID NO: 10 |
| TUS-protein | KLKIKRPVK | SEQ ID NO: 11 |
| Monopartite NLS Consensus | K-K/R-X-K/R | SEQ ID NO: 12 |
| Sequence | | |

Protein-Transduction Domain Sequences

| Name | Sequence | SEQ ID NO: |
|---|---|---|
| Tat 47-60 | YGRKKRRQRRRPPQ | SEQ ID NO: 13 |
| Penetratin | RQIKIWFQNRRMKWKK | SEQ ID NO: 14 |
| Transportan¹ | GWTLNSAGYLLGKINLKALAALAKKL | SEQ ID NO: 15 |
| Xentry, N-terminal region of the | LCLRPVG | SEQ ID NO: 16 |
| X-protein of the hepatitis B virus | | |
| Arginine₈₋₁₀ | RRRRRRRR (RR) | SEQ ID NO: 17 |
| Lysine₈₋₁₀² | KKKKKKKK (KK) | SEQ ID NO: 18 |
| MAP³ | KLALKLALKALKAALKLA | SEQ ID NO: 19 |
| Pep-1⁴ | KETWWETWWTEWSQPKKKRKV | SEQ ID NO: 20 |
| Pept1⁵ | PLILLRLLRGQF | SEQ ID NO: 21 |
| Pept2⁵ | PLIYLRLLRGQF | SEQ ID NO: 22 |
| IVV-14⁶ | KLWMRWYSPTTRRYG | SEQ ID NO: 23 |
| Amphiphilic model peptide⁷ | KLALKLALKALKAALKLA | SEQ ID NO: 24 |
| pVEC⁸ | LLIILRRRIRKQAHAHSK | SEQ ID NO: 25 |
| HRSV⁹ | RRIPNRRPRR | SEQ ID NO: 26 |
| PTD-5¹⁰ | RRQRRTSKLMKR | SEQ ID NO: 27 |

¹Pooga et al., FASEB J. 12: 67-77 (1998)
²Mi et al., J. Biol. Chem. 277(33): 30208-30218 (2002)
³Robbins et al., Cancer Res. 51: 3657-3662 (1991)
⁴Deshayes et al., Biochemistry 43(6): 1449-1457 (2004)
⁵Marks et al., J. Am. Chem. Soc. 133(23): 8995-9004 (2011)
⁶Kamide et al., Int. J. Mol. Med. 25(1): 41-51 (2010)
⁷Lindgren et al. Trend Pharmacol. Sci. 21(3): 99-103 (2000)
⁸Sidhu and Weiss, in Anticancer Drug Development, Baguley and Kerr, Ed., Academic Press 237-248 (2002)
⁹Langedijk et al, in Drug Transport(ers) and the Diseased Brain, International Congress Series, Elsevier 95-107 (2005)
¹⁰Mi et al., Mol. Ther. 2(4): 339-347 (2000)

In one embodiment, the NLS-Eomes-PTD polypeptide comprises residues 16-21 of SEQ ID NO: 7; residues 267-686 of SEQ ID NO: 2 or 4, and SEQ ID NO: 13. In another embodiment, each of the foregoing domains is separated by a glycine-serine linker. Various glycine serine linkers may be used and can comprise lengths of 4 to 20 amino acids. Typical linkers comprise repeats (e.g., 2, 3, or 4 repeats) of the sequence Gly-Gly-Gly-Gly-Ser (SEQ ID NO: 28). In one embodiment the glycine-serine linker has the amino acid the sequence Gly-Gly-Gly-Gly-Ser (SEQ ID NO: 28).

In one embodiment, the NLS-Eomes-PTD polypeptide is encoded by SEQ ID NO: 1. In another embodiment, the NLS-Eomes-PTD polypeptide has an amino acid sequence of SEQ ID NO: 2.

One embodiment described herein is a nucleotide sequence encoding a polypeptide, wherein the polypeptide comprises an Eomesodermin domain, and one or more nuclear localization signals and/or protein-transduction domain sequences. In one aspect, the Eomesodermin domain comprises an Eomesodermin transcription factor associated domain. In another aspect, the polypeptide comprises one or more nuclear localization signals, the Eomesodermin transcription factor associated domain, and one or more protein-transduction domains, each optionally separated by a glycine-serine linker. In one aspect, the polypeptide has the structure: NLS-GL-Eomes-TFAD-GL-PTD; PTD-GL-Eomes-TFAD-GL-NLS; PTD-GL-Eomes-TFAD; or NLS-GL-Eomes-TFAD; wherein NLS is a nuclear localization signal; GL is an optional glycine-serine linker; Eomes-TAD is an Eomesodermin transcription factor associated domain, PTD is a protein-transduction domain. In another aspect, the polypeptide further comprises an affinity tag. In another aspect, the affinity tag comprises a 6×-histidine tag. In another aspect, the Eomesodermin transcription factor associated domain comprises SEQ ID NO: 2 or 4. In another aspect, the Eomesodermin transcription factor associated domain comprises residues 267-686 of SEQ ID NO: 2 or 4. In another aspect, the nuclear localization signal comprises one or more of SEQ ID NO: 7-11 and the protein-transduction domain comprises one or more of SEQ ID NO: 13-27. In another aspect, the nuclear localization signal comprises residues 16-21 of SEQ ID NO: 7; the protein-transduction domain comprises residues 1-11 of SEQ ID NO: 13; and the glycine-serine linker comprises SEQ ID NO: 28. In another aspect, the nucleotide sequence has 85% to 99% identity to SEQ ID NO: 1. In another aspect, the nucleotide sequence is SEQ ID NO: 1.

Another embodiment described herein is a polynucleotide vector comprising a nucleotide sequence described herein.

Another embodiment described herein is a cell comprising one or more nucleotide sequences of described herein or a polynucleotide described herein.

Another embodiment described herein is a polypeptide encoded by the nucleotide sequence described herein.

Another embodiment described herein is a polypeptide encoded by the nucleotide sequence described herein, wherein the polypeptide has 85% to 99% identity to SEQ ID NO: 2.

A polypeptide encoded by the nucleotide sequence described herein, wherein the polypeptide is SEQ ID NO: 2.

Another embodiment described herein is a research tool comprising a polypeptide encoded by the nucleotide sequence described herein.

Another embodiment described herein is a therapeutic reagent comprising a polypeptide encoded by the nucleotide sequence described herein.

Another embodiment described herein is a means or method for manufacturing the nucleotide sequence described herein or a polypeptide encoded by the nucleotide sequence described herein, the process comprising: transforming or transfecting a cell with a nucleic acid as described herein; growing the cells; optionally, harvesting the cells and isolating quantities of the nucleotide sequence described herein; inducing expression of a polypeptide encoded by the nucleotide sequence described herein; harvesting the cells; and isolating and purifying the polypeptide.

Another embodiment is a nucleotide sequence described herein or a polypeptide encoded by the nucleotide sequence described herein, each produced by the means or methods described herein/

Another embodiment described herein is a therapeutic polypeptide comprising the amino acid sequence of SEQ ID NO: 2.

Another embodiment described herein is a method for rejuvenating exhausted natural killer cells, the method comprising: obtaining exhausted natural killer cells (ExNK); contacting the natural killer cells with a quantity of a polypeptide comprising one or more nuclear localization signals, an Eomesodermin transcription factor associated domain, and one or more protein-transduction domains; incubating the ExNK and polypeptide for a period of time sufficient for the polypeptide to be uptaken by the cells (transduced); and allowing the cells to incubate for a period of time sufficient for the polypeptide to rejuvenate the ExNKs.

Another embodiment described herein is a method for treating cancer, the method comprising: (a) administering a therapeutically effective amount of polypeptide comprising one or more nuclear localization signals, an Eomesodermin transcription factor associated domain, and one or more protein-transduction domains to a subject in need thereof; or (b) contacting autologous exhausted natural killer cells isolated from a subject with a therapeutically effective amount of polypeptide comprising one or more nuclear localization signals, an Eomesodermin transcription factor associated domain, and one or more protein-transduction domains; permitting the cells to rejuvenate; and administering the cells to a subject in need thereof;

Another embodiment described herein is the use of a polypeptide comprising one or more nuclear localization signals, an Eomesodermin transcription factor associated domain, and one or more protein-transduction domains for the rejuvenation of exhausted natural killer cells or the treatment of a subject in need thereof.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% or 100% identical to (a) nucleotide sequences, or degenerate, homologous, or codon-optimized variants thereof, encoding polypeptides having the amino acid sequences in SEQ ID NO: 2; (b) nucleotide sequences, or degenerate, homologous, or codon-optimized variants thereof, encoding polypeptides having the amino acid sequences in SEQ ID NO:2; and (c) nucleotide sequences capable of hybridizing to the complement of any of the nucleotide sequences in (a) or (b) above and capable of expressing functional polypeptides of amino acid sequences in SEQ ID NO: 2.

By a polynucleotide having a nucleotide sequence at least, for example, 90-99% “identical” to a reference nucleotide sequence encoding a recombinant Eomes polypetide is intended that the nucleotide sequence of the polynucleotide be identical to the reference sequence except that the polynucleotide sequence can include up to about 10 to 1 point mutations, additions, or deletions per each 100 nucleotides of the reference nucleotide sequence encoding a recombinant Eomes polypetide.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2: 4 82-489 (1981). This algorithm can be extended to use with peptide sequences using the scoring matrix developed by Dayhoff, Atlas of Protein Sequences and Structure, M. O. Dayhoff ed., 5 suppl. 3: 353-358, National Biomedical Research Foundation, Washington, D.C., USA, and normalized by Gribskov, Nucl. Acids Res. 14(6): 6745-6763 (1986).

For example, due to the degeneracy of the genetic code, one having ordinary skill in the art will recognize that a large number of the nucleic acid molecules having a sequence at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequence shown in SEQ ID NO: 1, or degenerate, homologous, or codon-optimized variants thereof, will encode a recombinant Eomes polypetide.

The polynucleotides described herein include those encoding mutations, variations, substitutions, additions, deletions, and particular examples of the polypeptides described herein. For example, guidance concerning how to make phenotypically silent amino acid substitutions is provided in Bowie, J. U. et al., “Deciphering the Message in Protein Sequences: Tolerance to Amino Acid Substitutions,” Science 247: 1306-1310 (1990), wherein the authors indicate that proteins are surprisingly tolerant of amino acid substitutions.

Thus, fragments, derivatives, or analogs of the polypeptides of SEQ ID NO: 2 can be (i) ones in which one or more of the amino acid residues (e.g., 1, 2, 3, 4, 5, 7, 8, 9, 10, 11, 12, 13, 14, 15, 16, 17, 18, 19, 20, 25, 30, 35, 40, 45, or 50 residues, or even more) are substituted with a conserved or non-conserved amino acid residue (preferably a conserved amino acid residue). Such substituted amino acid residues may or may not be one encoded by the genetic code, or (ii) ones in which one or more of the amino acid residues includes a substituent group (e.g., 1, 2, 3, 4, 5, 7, 8, 9, 10, 15, 20, 25, 30, 35, 40, 45, or 50 residues or even more), or (iii) ones in which the mature polypeptide is fused with another polypeptide or compound, such as a compound to increase the half-life of the polypeptide (for example, polyethylene glycol), or (iv) ones in which the additional amino acids are fused to the mature polypeptide, such as an IgG Fc fusion region peptide or leader or secretory sequence or a sequence which is employed for purification of the mature polypeptide or a proprotein sequence. Such fragments, derivatives, and analogs are deemed to be within the scope of those skilled in the art from the teachings herein.

In addition, fragments, derivatives, or analogs of the polypeptides of SEQ ID NO: 2 can be substituted with one or more conserved or non-conserved amino acid residue (preferably a conserved amino acid residue). In some cases these polypeptides, fragments, derivatives, or analogs thereof will have a polypeptide sequence at least 50%, 55%, 60%, 65%, 70%, 75%, 80%, 85%, 90%, 91%, 92%, 93%, 94%, 95%, 96%, 97%, 98%, 99% or 100% identical to the polypeptide sequence shown in SEQ ID NO: 2 and will comprise functional or non-functional proteins or enzymes. Similarly, additions or deletions to the polypeptides can be made either at the N- or C-termini or within non-conserved regions of the polypeptide (which are assumed to be non-critical because they have not been photogenically conserved).

As described herein, in many cases the amino acid substitutions, mutations, additions, or deletions are preferably of a minor nature, such as conservative amino acid substitutions that do not significantly affect the folding or activity of the protein or additions or deletions to the N- or C-termini. Of course, the number of amino acid substitutions, additions, or deletions a skilled artisan would make depends on many factors, including those described herein. Generally, the number of substitutions, additions, or deletions for any given polypeptide will not be more than about 100, 90, 80, 70, 60, 50, 40, 30, 25, 20, 19, 18, 17, 16, 15, 14, 13, 12, 11, 10, 9, 8, 7, 5, 6, 4, 3, 2, or 1.

It will be apparent to one of ordinary skill in the relevant art that suitable modifications and adaptations to the compositions, formulations, methods, processes, apparata, assemblies, and applications described herein can be made without departing from the scope of any embodiments or aspects thereof. The compositions, apparata, assemblies, and methods provided are exemplary and are not intended to limit the scope of any of the disclosed embodiments. All the various embodiments, aspects, and options disclosed herein can be combined in any variations or iterations. The scope of the compositions, formulations, methods, apparata, assemblies, and processes described herein include all actual or potential combinations of embodiments, aspects, options, examples, and preferences described herein. The compositions, formulations, apparata, assemblies, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. All patents and publications cited herein are incorporated by reference herein for the specific teachings thereof.

Various embodiments and aspects of the inventions described herein are summarized by the following clauses:
Clause 1. A nucleotide sequence encoding a polypeptide, wherein the polypeptide comprises an Eomesodermin domain, and one or more nuclear localization signals and/or protein-transduction domain sequences.
Clause 2. The nucleotide sequence of clause 1, wherein the Eomesodermin domain comprises an Eomesodermin transcription factor associated domain.
Clause 3. The nucleotide sequence of clause 1 or 2, wherein the polypeptide comprises one or more nuclear localization signals, the Eomesodermin transcription factor associated domain, and one or more protein-transduction domains, each optionally separated by a glycine-serine linker.
Clause 4. The nucleotide sequence of any one of clauses 1-3, wherein the polypeptide has the structure:
   NLS-GL-Eomes-TFAD-GL-PTD;
   PTD-GL-Eomes-TFAD-GL-NLS;
   PTD-GL-Eomes-TFAD; or
   NLS-GL-Eomes-TFAD;
wherein NLS is a nuclear localization signal; GL is an optional glycine-serine linker; Eomes-TAD is an Eomesodermin transcription factor associated domain, PTD is a protein-transduction domain.
Clause 5. The nucleotide sequence of any one of clauses 1-4, wherein the polypeptide further comprises an affinity tag.
Clause 6. The nucleotide sequence of any one of clauses 1-5, wherein the affinity tag comprises a 6×-histidine tag.
Clause 7. The nucleotide sequence of any one of clauses 1-6, wherein the Eomesodermin transcription factor associated domain comprises SEQ ID NO: 2 or 4.
Clause 8. The nucleotide sequence of any one of clauses 1-7, wherein the Eomesodermin transcription factor associated domain comprises residues 267-686 of SEQ ID NO: 2 or 4.
Clause 9. The nucleotide sequence of any one of clauses 1-8, wherein the nuclear localization signal comprises one or more of SEQ ID NO: 7-11 and the protein-transduction domain comprises one or more of SEQ ID NO: 13-27.
Clause 10. The nucleotide sequence of any one of clauses 1-9, wherein the nuclear localization signal comprises residues 16-21 of SEQ ID NO: 7; the protein-transduction domain comprises residues 1-11 of SEQ ID NO: 13; and the glycine-serine linker comprises SEQ ID NO: 28.
Clause 11. The nucleotide sequence of any one of clauses 1-10, wherein the nucleotide sequence has 85% to 99% identity to SEQ ID NO: 1.
Clause 12. The nucleotide sequence of any one of clauses 1-11, wherein the nucleotide sequence is SEQ ID NO: 1.
Clause 13. A polynucleotide vector comprising a nucleotide sequence of any one of clauses 1-12.
Clause 14. A cell comprising one or more nucleotide sequences of clause 1-12 or a polynucleotide vector of clause 13.
Clause 15. A polypeptide encoded by the nucleotide sequence of any one of clauses 1-12.
Clause 16. A polypeptide encoded by the nucleotide sequence of any one of clauses 1-12, wherein the polypeptide has 85% to 99% identity to SEQ ID NO: 2.
Clause 17. A polypeptide encoded by the nucleotide sequence of any one of clauses 1-12, wherein the polypeptide is SEQ ID NO: 2.
Clause 18. A research tool comprising a polypeptide encoded by the nucleotide sequence of any one of clauses 1-12.
Clause 19. A therapeutic reagent comprising a polypeptide encoded by the nucleotide sequence of any one of clauses 1-12.
Clause 20. A means or method for manufacturing the nucleotide sequence of any one of clauses 1-12 or a polypeptide encoded by the nucleotide sequence of any one of clauses 1-12, the process comprising: transforming or transfecting a cell with a nucleic acid comprising the nucleotide sequence of clause 1; growing the cells; optionally, harvesting the cells and isolating quantities of the nucleotide sequence of any one of clauses 1-12; inducing expression of a polypeptide encoded by the nucleotide sequence of clause 1-12; harvesting the cells; and isolating and purifying the polypeptide.
Clause 21. The nucleotide sequence of any one of clauses 1-12 or a polypeptide encoded by the nucleotide sequence of any one of clauses 1-12, each produced by the means or method of clause 20.
Clause 22. A therapeutic polypeptide comprising the amino acid sequence of SEQ ID NO: 2.
Clause 23. A method for rejuvenating exhausted natural killer cells, the method comprising:
   obtaining exhausted natural killer cells (ExNK);
   contacting the natural killer cells with a quantity of a polypeptide comprising one or more nuclear localization signals, an Eomesodermin transcription factor associated domain, and one or more protein-transduction domains;
   incubating the ExNK and polypeptide for a period of time sufficient for the polypeptide to be uptaken by the cells (transduced); and
   allowing the cells to incubate for a period of time sufficient for the polypeptide to rejuvenate the ExNKs.
Clause 24. A method for treating cancer, the method comprising:
   (a) administering a therapeutically effective amount of polypeptide comprising one or more nuclear localization signals, an Eomesodermin transcription factor associated domain, and one or more protein-transduction domains to a subject in need thereof; or
   (b) contacting autologous exhausted natural killer cells isolated from a subject with a therapeutically effective amount of polypeptide comprising one or more nuclear localization signals, an Eomesodermin transcription factor associated domain, and one or more protein-transduction domains; permitting the cells to rejuvenate; and administering the cells to a subject in need thereof;
Clause 25. Use of a polypeptide comprising one or more nuclear localization signals, an Eomesodermin transcription factor associated domain, and one or more protein-transduction domains for the rejuvenation of exhausted natural killer cells or the treatment of a subject in need thereof.

### EXAMPLES

### Example 1

### Eomes Preparation

An exemplary recombinant Eomes protein was developed by combining an SV40 Large T-antigen Nuclear Localization Signal (NLS; residues 16-21 of SEQ ID NO: 7) with the Eomes polypeptide sequence (residues 267-686 of SEQ ID NO: 2), and a cytoplasm penetration peptide, (e.g., HIV transactivating transcriptional activator, TAT; residues 1-11 of SEQ ID NO: 13). The domains are linked by glycine-serine linkers having the amino acid the sequence Gly-Gly-Gly-Gly-Ser (SEQ ID NO: 28). The penetration peptide sequences are used to direct the Eomes protein into cells. Any cytoplasm penetration peptide or nuclear localization signals can be used (see Table 2) but using both cytoplasm and nucleus penetration peptides are desirable, because Eomes needs to enter the nucleus to works as a transcriptional factor. The transcriptional factor region of human Eomes sequence was obtained from amino acid residues 267-686 of NCBI reference sequence NP_005433.2 (human eomesodermin homolog isoform 2). The first amino acid was changed to a methionine to facilitate expression in E. coli. Polyglycine amino acid spacers were added to between each peptide domain to permit them to function independently.

The nucleotide and amino acid sequences for the recombinant Eomes polynucleotide construct, Eomes polypeptide, and plasmid DNA are provided:

Eomes Polynucleotide Sequence (SEQ ID NO: 1)
ATGCCTAAAAAGAAAAGGAAGGTTGGTGGGGGAGGATCAGCTCACGTTTATCTGTGCAACCGCCCGCTGTGGCTGA
AGTTCCATCGTCACCAGACCGAGATGATCATTACCAAACAAGGTCGTCGTATGTTCCCGTTTCTGAGCTTCAACAT
CAACGGCCTGAACCCGACCGCGCACTACAACGTGTTCGTTGAAGTGGTTCTGGCGGACCCGAACCACTGGCGTTTT
CAGGGTGGCAAATGGGTTACCTGCGGCAAGGCGGATAACAACATGCAAGGCAACAAGATGTATGTGCACCCGGAGA
GCCCGAACACCGGTAGCCACTGGATGCGTCAGGAAATTAGCTTTGGCAAGCTGAAACTGACCAACAACAAAGGCGC
GAACAACAACAACACCCAGATGATCGTTCTGCAAAGCCTGCACAAGTACCAACCGCGTCTGCACATTGTGGAGGTT
ACCGAAGACGGCGTGGAGGATCTGAACGAACCGAGCAAAACCCAGACCTTCACCTTTAGCGAAACCCAATTCATCG
CGGTTACCGCGTACCAGAACACCGACATCACCCAACTGAAAATTGATCACAACCCGTTCGCGAAGGGTTTTCGTGA
CAACTACGATAGCAGCCACCAGATTGTGCCGGGTGGCCGTTATGGCGTTCAGAGCTTCTTCCCGGAGCCGTTTGTG
AACACCCTGCCGCAGGCGCGTTACTATAACGGTGAACGTACCGTTCCGCAAACCAACGGCCTGCTGAGCCCGCAGC
AAAGCGAGGAAGTGGCGAACCCGCCGCAGCGTTGGCTGGTGACCCCGGTTCAGCAACCGGGTACCAACAAACTGGA
CATCAGCAGCTACGAGAGCGAATATACCAGCAGCACCCTGCTGCCGTACGGTATTAAGAGCCTGCCGCTGCAGACC
AGCCATGCGCTGGGCTACTATCCGGATCCGACCTTTCCGGCGATGGGGGGTTGGGGTGGCCGTGGCAGCTATCAGC
GTAAAATGGCGGCGGGTCTGCCGTGGACCAGCCGTACCAGCCCGACCGTGTTTAGCGAGGACCAACTGAGCAAGGA
AAAAGTTAAAGAGGAGATCGGTAGCAGCTGGATTGAAACCCCGCCGAGCATCAAAAGCCTGGACAGCAACGATAGC
GGCGTGTACACCAGCGCGTGCAAGCGTCGTCGTCTGAGCCCGAGCAACAGCAGCAACGAGAACAGCCCGAGCATCA
AGTGCGAAGACATTAACGCGGAGGAATATAGCAAAGATACCAGCAAGGGTATGGGTGGCTACTATGCGTTTTACAC
CACCCCGGGTGGCGGTGGCAGCTATGGCCGTAAGAAGCGTCGTCAGCGTCGTCGCGGGGGGGGCGGCAGCCATCAC
CATCACCATCACGGTTAATGA
Eomes Polypeptide Sequence (SEQ ID NO: 2)
MPKKKRKV-GGGGS-AHVYLCNRPLWLKFHRHQTEMIITKQGRRMFPFLSFNINGLNPTAHYNVEVEVVLADPNHW
RFQGGKWVTCGKADNNMQGNKMYVHPESPNTGSHWMRQEISFGKLKLTNNKGANNNNTQMIVLQSLHKYQPRLHIV
EVTEDGVEDLNEPSKTQTFTFSETQFIAVTAYQNTDITQLKIDHNPEAKGFRDNYDSSHQIVPGGRYGVQSFFPEP
FVNTLPQARYYNGERTVPQTNGLLSPQQSEEVANPPQRWLVTPVQQPGINKLDISSYESEYTSSTLLPYGIKSLPL
QTSHALGYYPDPTFPAMAGWGGRGSYQRKMAAGLPWTSRTSPTVFSEDQLSKEKVKEEIGSSWIETPPSIKSLDSN
DSGVYTSACKRRRLSPSNSSNENSPSIKCEDINAEEYSKDTSKGMGGYYAFYTTP-GGGGS-YGRKKRRQRRR-GG
GGS-HHHHHHG
Eomes Plasmid DNA Sequence (SEQ ID NO: 30)
Key:
NLS-spacer-Eomes-spacer-TAT-spacer-6xHis

Eomes Expression in E. coli

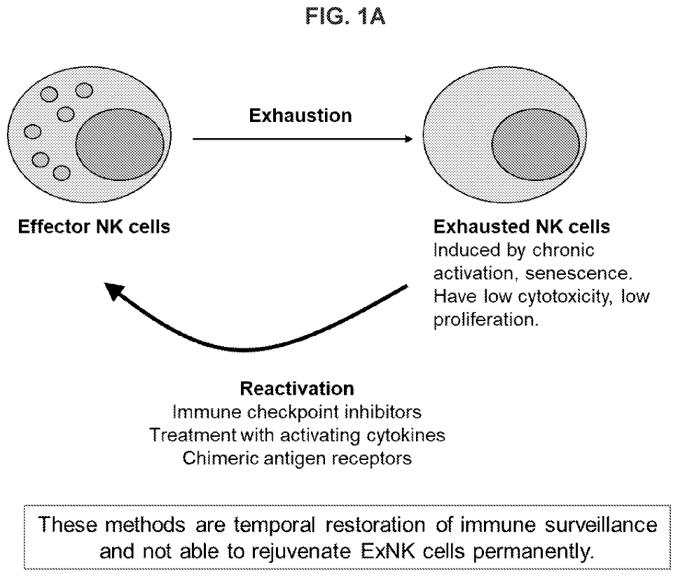

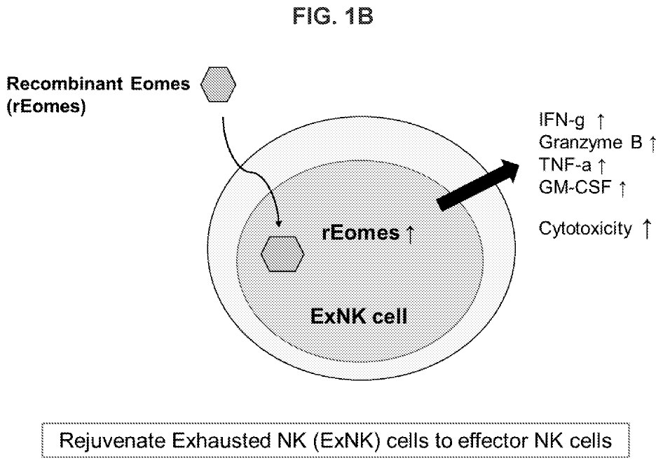

The recombinant Eomes protein (52 kDa) was expressed in BL21 E. coli, the cells were lysed, and the polypeptide purified using a nickle column. The bicinchoninic acid (BCA) assay (Thermo Fisher) was used for protein quantitation. Eomes expression was confirmed by SDS gel image that protein molecular weight is around 50 kDa (FIG. 1). Coomassie staining showed a band at around 50 kDa and indicated that Eomes was expressed.

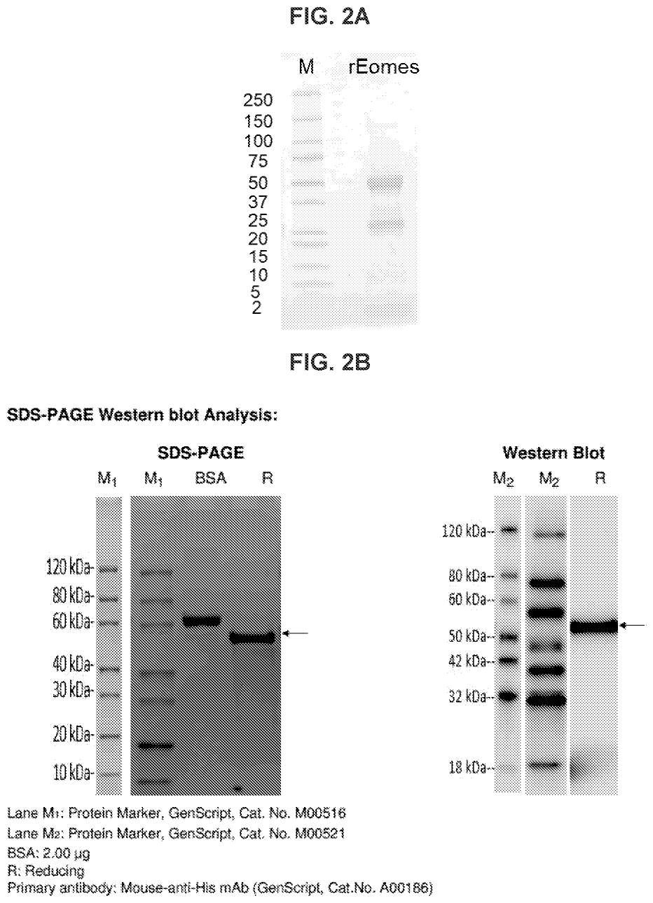

The purified protein was analyzed and confirmed using SDS-PAGE and western blot analysis (FIG. 2). SDS-PAGE showed one band near 52 kDa that indicated that recombinant Eomes (52 kDa) was expressed and purified with high purity. Western blot analysis confirmed that the His-tagged Eomes protein was expressed by showing a single band around 52 kDa as identified with an anti-His tag antibody.

### Example 2

### In Vitro Eomes Experiments

### Cells and Culture Media

Human NK-92 (NK) cells were obtained from ATCC® and cultured in X-vivo 15 media (Lonza Bioscience) with 10% FBS, 1% pencillin/streptomycin, and 250 U/mL of interlukin-2 (IL-2). To generate exhausted NK (ExNK) cells, NK cells were continuously cultured over 70 passages. To verify the exhausted state, expression levels of the inhibitory biomarker NKG2A were compared between ExNK and fresh NK cells using flow cytometry analyses with an PE-anti-NKG2A antibody label. Once the expression level of NKG2A was higher than normal NK cells, ExNK cells were utilized for further in vitro and in vivo studies.

### Assays of In Vitro Anti-Cancer Effects

To observe changes on the anti-cancer effect of NK and ExNK cells through Eomes transduction, cancer cell death rate and cytokine secretion were analyzed. Cancer cells were pre-labeled with 25 μM CellTracker Blue CMAC and seeded at 1×10⁴ cells/well in 48-well plates, 24 hours prior to commencing the experiment. The cells were then incubated with NK, ExNK, Eomes-NK, or Eomes-ExNK cells according to an effector:target (E:T) ratio, where effector and target referred immune cells and cancer cells, respectively. After the 4 h inoculation, cells and supernatant were harvested, and the cells were labeled with the Annexin V Alexa Fluor 488 and propidium iodide (PI) kit (Thermo Fisher). Cancer cell death was analyzed Annexin V- and PI-stained cells in the CellTracker Blue-stained population by flow cytometry (BD LSRII). The harvested supernatant was stored at −20° C. for subsequent ELISA assays of the cytokines IFN-γ, TNF-α, granzyme B, and GM-CSF, which are representative cytokines of NK cells.

### Confirmation of Eomes Uptake Using Fluorescence Microscopy

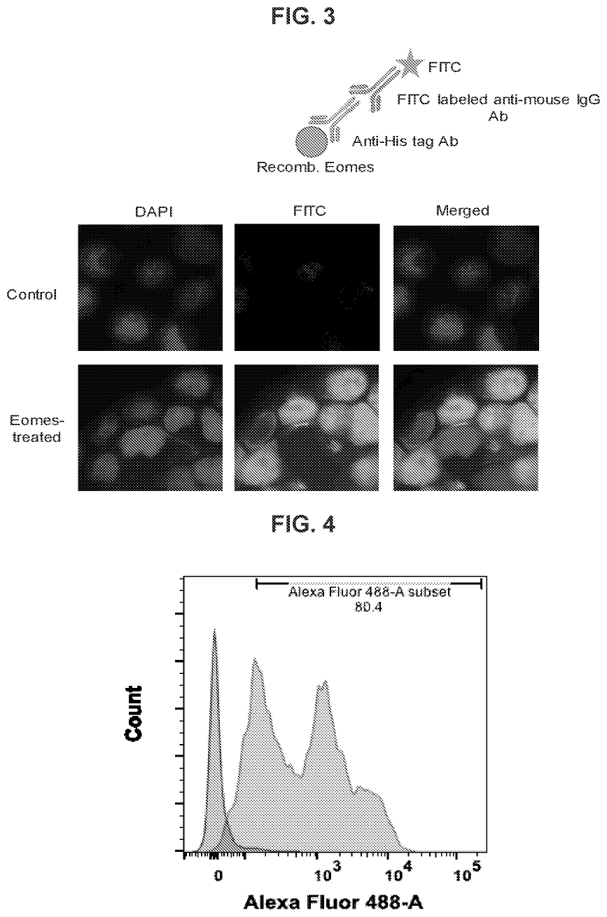

Before optimizing the Eomes transduction condition, Eomes uptake was confirmed by fluorescent microscopy (FIG. 3). From various protein transduction conditions, one transduction condition (2 μM, 3 h protein co-incubation) was chosen. Then the NK cells were fixed and stained with anti-His tag and FITC-labeled anti-mouse IgG antibodies. Nuclei were stained with DAPI as counter staining. Recombinant Eomes has a His-tag and this motif is not naturally expressed in mammalian cells, therefore, His-tag-positive cells indicate recombinant Eomes-internalized cells. Eomes transduced NK (Eomes-NK) cells had FITC-positive cells while control (non-treated cells) had negligible FITC signal. This indicated that Eomes transduced into NK cells.

### Confirmation of Eomes Uptake Using Flow Cytometry

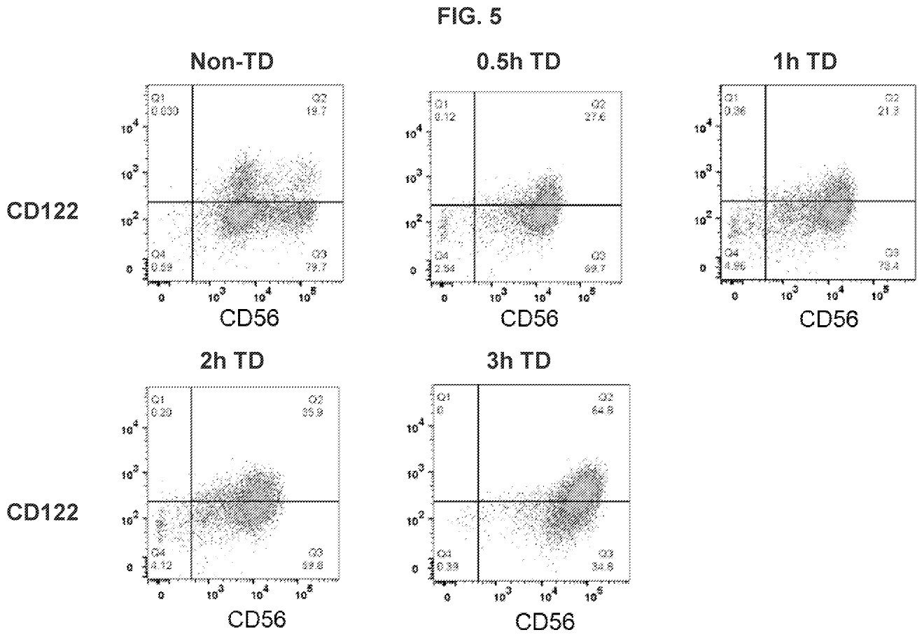

Recombinant Eomes was transduced into NK cells using the same condition as above (2 μM and 3 h transduction time). Then both Eomes-transduced- and non-transduced-NK cells were collected and immediately permeabilized/stained with anti-His tag antibody and Alexa Fluor-488-labled anti-mouse IgG antibody. The fluorescent intensity of cells was measured by flow cytometry (BD LSRII) (FIG. 4). In FIG. 4, grey colored histogram indicates control which is Eomes non-treated NK cells, and red colored histogram indicates Eomes-NK cells. Once the cells have Alexa Fluor-488 signal, it refers that recombinant Eomes containing His tag exists in cells. Recombinant Eomes-NK cells (red histogram) had Alexa fluor-488 signals compared to control (grey histogram).

### Optimization of Eomes Transduction Time

Transduction conditions evaluated uptake amount and cellular changes caused by Eomes. The Eomes transduction time was optimized through phenotypical changes. The changes were confirmed by flow cytometry. Eomes has a co-upregulated relation with CD122. Therefore, NK cells from various condition of Eomes transduction were stained with APC-anti-CD56 and FITC-anti-CD122 antibodies (FIG. 5). The Eomes amount was fixed at 2 μM, the same concentration that was used for uptake analysis. The cells were harvested immediately after transduction and stained with the antibodies. Among various transduction time, 3 h transduction time had the greatest effect on CD122. A transduction time of 3 h was therefore optimal.

### Priming Eomes-Transduced NK Cells (Eomes-NK)

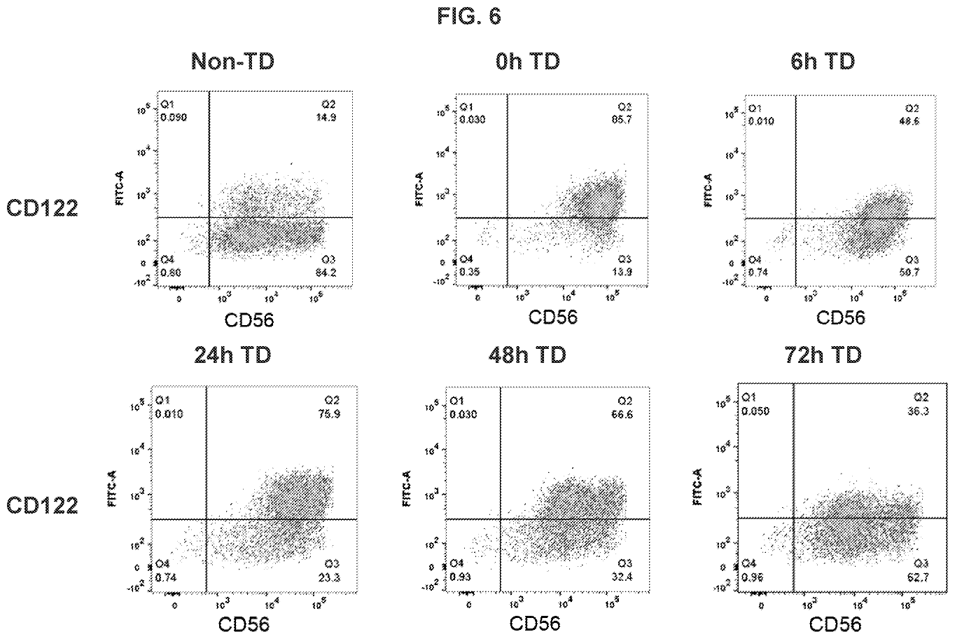

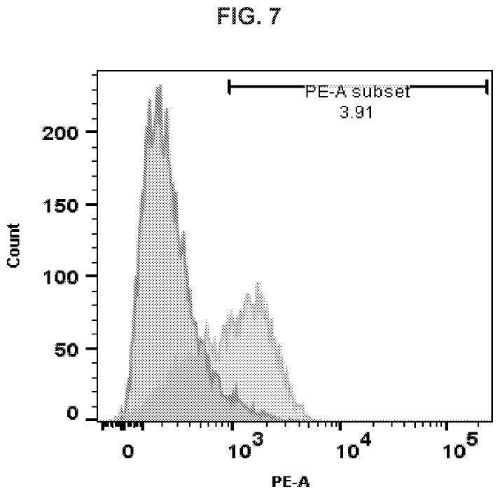

After Eomes was transduced into NK cells, it was determined whether Eomes-priming time was needed or not. NK cells were additionally incubated for various time after 3 h transduction of 2 μM Eomes. Once the priming time was complet, the cells were harvested and stained with APC-anti-CD56 and FITC-anti-CD122 antibodies (FIG. 6). Fluorescent intensity was measured by flow cytometry (BD LSRII). CD122 expression was decreased for 6 h of priming time and increased back at 24 h. Then the expression slowly weakened until 72 h. If Eomes-NK cells were used with no priming time, Eomes-related changes would not be effective for a day. However, Eomes-NK cells with 24 h priming time could bring effective Eomes-related changes for at least a day and even two days. A 24 h of priming time after transduction was chosen for Eomes-NK cells.

### Optimization of Eomes Amount for Transduction

With the optimal transduction and priming time condition were analysed as described above, the optimal amount of Eomes was analyzed further. Before optimization of transduction amount, normal NK cells and exhausted NK cells (ExNK) were prepared for an Eomes-mediated rejuvenation experiment.

ExNK cells were generated through prolonged cultivation of NK cells (more than 70 passages). Judge et al., Front. Cell. Infect. Microbiol. 10: 49 (2020) doi: 10.3389/fcimb.2020.00049. Generation of ExNK cells were confirmed by comparison of CD56 and NKG2A expression using flow cytometry. ExNK cells had similar CD56 expression and stronger expression of NKG2A compared to NK cells. NKG2A is an inhibitory marker of NK cells, and the high expression of NKG2A indicates that ExNK cells are either exhausted or have inhibited cytolytic activity.

NK and ExNK cells were treated with various amounts of Eomes with 3 h of transduction time and 24 h priming time. After Eomes transduction and priming were done, the cells were collected and labeled with APC-anti-CD56, FITC-anti-CD122, PE-anti-NKG2A antibodies. Phenotypical changes were monitored through flow cytometry (Tables 3 and 4). Interestingly, NK cells had increased NKG2A expression after Eomes transduction, while ExNK cells had decreased NKG2A expression after Eomes transduction. These results indicated that abnormal Eomes expression may impaired NK cell function, and ExNK cells may rejuvenate through decreased NKG2A expression. For ExNK cells, CD122 expression was saturated at 0.5 μM Eomes. Therefore, the optimal Eomes amount for transduction was fixed at 0.5 μM. The optimal transduction conditions (0.5 μM Eomes, 3 h transduction, 24 h priming) was applied for all in vitro and in vivo assays described below.

TABLE 3
Phenotypic Changes in NK by Eomes Transduction

| Eomes (μM) | CD56 | CD122 | CD56 + CD122 | NKG2A |
|---|---|---|---|---|
| Control | 5.46 | 5.6 | 0.11 | 18.4 |
| 0.1 | 41.8 | 20.6 | 0.87 | 65.7 |
| 0.2 | 15.4 | 36/3 | 0.59 | 54.4 |
| 0.5 | 15.7 | 28.4 | 0.4 | 50.9 |
| 0.7 | 25.4 | 18.0 | 0.7 | 60.4 |
| 1.0 | 9.18 | 20.9 | 0.33 | 59 |
| 1.5 | 24.8 | 23.6 | 1.17 | 54.4 |
| 2.0 | 18.4 | 11.9 | 0.48 | 42.4 |

TABLE 4
Phenotypic Changes in ExNK by Eomes Transduction

| Eomes (μM) | CD56 | CD122 | CD56 + CD122 | NKG2A |
|---|---|---|---|---|
| Control | 5.29 | 5.38 | 0.24 | 27.4 |
| 0.1 | 9.77 | 13.2 | 1.36 | 6.57 |
| 0.2 | 5.19 | 13.5 | 1.15 | 4.02 |
| 0.5 | 6.56 | 51.2 | 2.21 | 6.75 |
| 0.7 | 6.34 | 52.3 | 2.72 | 8.46 |
| 1.0 | 9.18 | 38.5 | 3.73 | 9.86 |
| 1.5 | 3.35 | 50.5 | 1.48 | 5.64 |
| 2.0 | 2.72 | 42.7 | 1.6 | 4.62 |
| 2 μM + 24 h Transduction | 12.2 | 6.87 | 0.66 | 10.8 |

While NK cells had increased NKG2A after Eomes transduction, ExNK cells had decreased NKG2A expression after Eomes transduction. CD122 expression is saturated at 0.5 μM Eomes.

### Example 3

### In Vitro Anti-Cancer Activity of Eomes-ExNK on Various Cancer Cell Types

These results were obtained before optimizing the amount of Eomes. Therefore, transduction conditions were 2 μM Eomes for 3 h transduction and 24 h priming, and the cells were analyzed. ExNK and Eomes-ExNK cells were analyzed for cancer cell death rate and cytokine secretion. Effector: Target (E:T) ratio, where effect was ExNK or Eomes-ExNK cell and target was cancer cell, was fixed at 5:1. Cancer cells were pre-labeled with cell-tracker prior to co-incubation with ExNK cells to distinguish them from ExNK cells. Cancer cells and ExNK/Eomes-ExNK cells were co-incubated for 4 hours, then the cells and supernatants were separately harvested for further analysis. The cells were stained with cell death kit (Annexin V/PI; annexin V stains apoptotic dead cells and PI stains dead cells including necrosis) and analyzed using flow cytometry. Cell-tracker labeled cells were selected and Annexin V positive cells were analyzed to measure only dead cancer cells; this represented the cell death percentage. These results showed Eomes-ExNK had stronger anti-cancer efficacy than ExNK cells on various cancer cell types. Supernatants were analyzed for anti-cancer cytokine ELISA. Eomes-ExNK cells had increased cytokine secretion compared to ExNK cells. These results indicated that Eomes recovered anti-cancer activity of ExNK cells, and Eomes-ExNK cells could be used for treatment of various types of cancer. MDA-MB-231 cells, triple negative breast cancer cells, were used for further studies.

In Vitro Cytotoxicity with Various E:T Ratios

In vitro anti-cancer activity was analyzed at various E:T ratios (0, 1, 5, 10 and 20). According to the optimal transduction condition, Eomes-NK and Eomes-ExNK cells were prepared by transduction of 0.5 μM Eomes for 3 h and additional 24 hours incubation for stabilization after transduction. MDA-MB-231 cells were labeled with cell tracker blue prior to seeding on 48-well plates. On next day, MDA-MB-231 cells were co-incubated with NK cells, Eomes-NK cells, ExNK cells, or Eomes-ExNK cells for 4 h. Superatant media and cells were separately harvested. The supernatant was utilized for cytokine ELISA and the cells were analyzed for cell death with flow cytometry as above. Cell death and cytokine ELISA were analyzed to confirm increased cytotoxicity according to E:T ratio and whether Eomes-ExNK cells get increased cytotoxicity as much as NK cells.

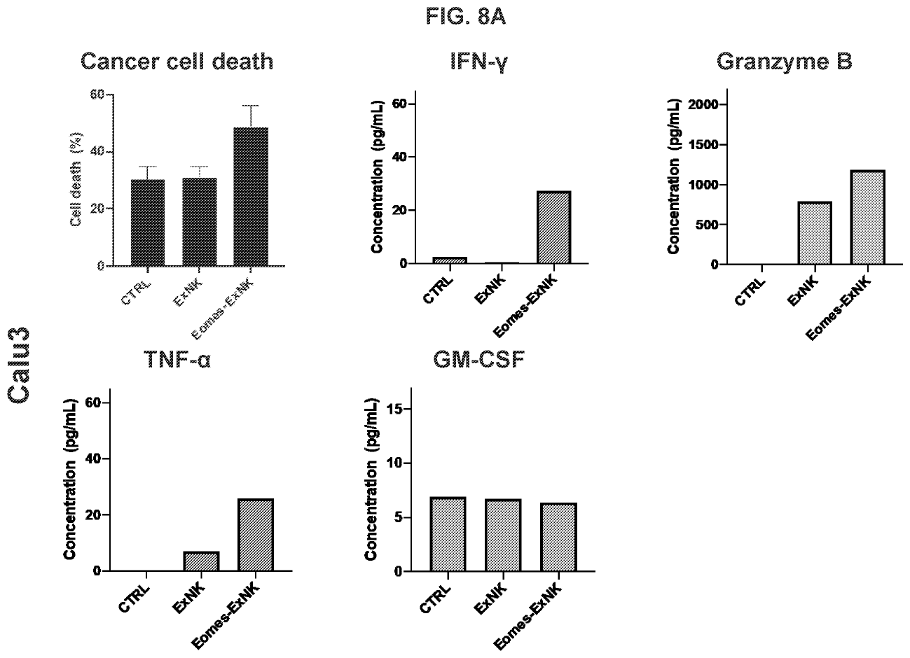

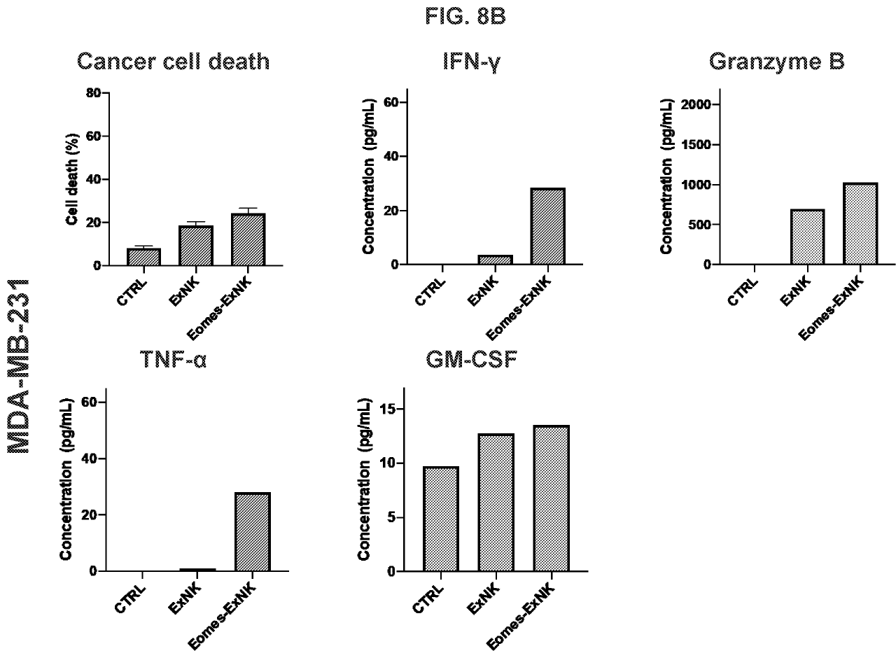

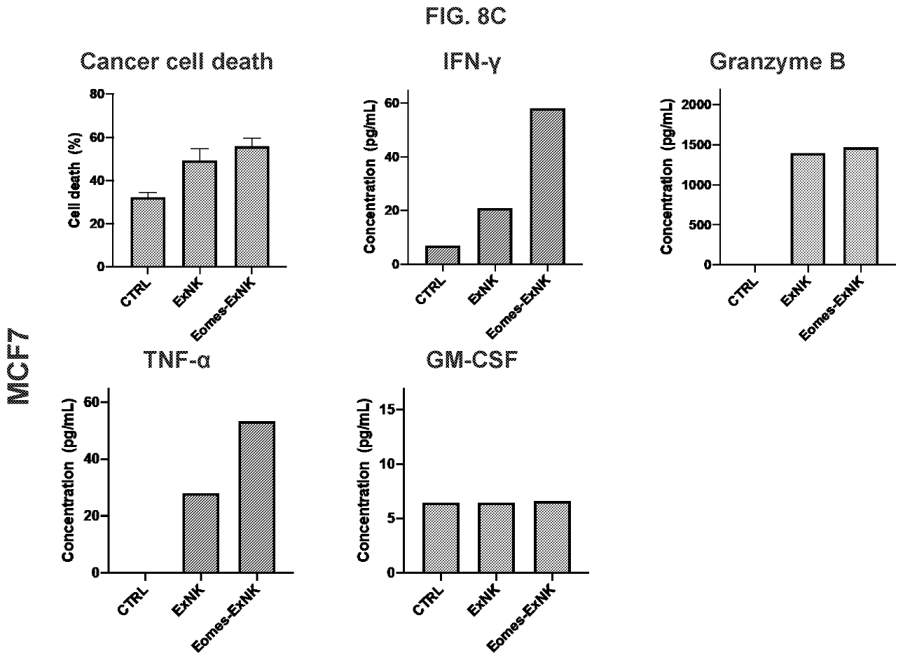

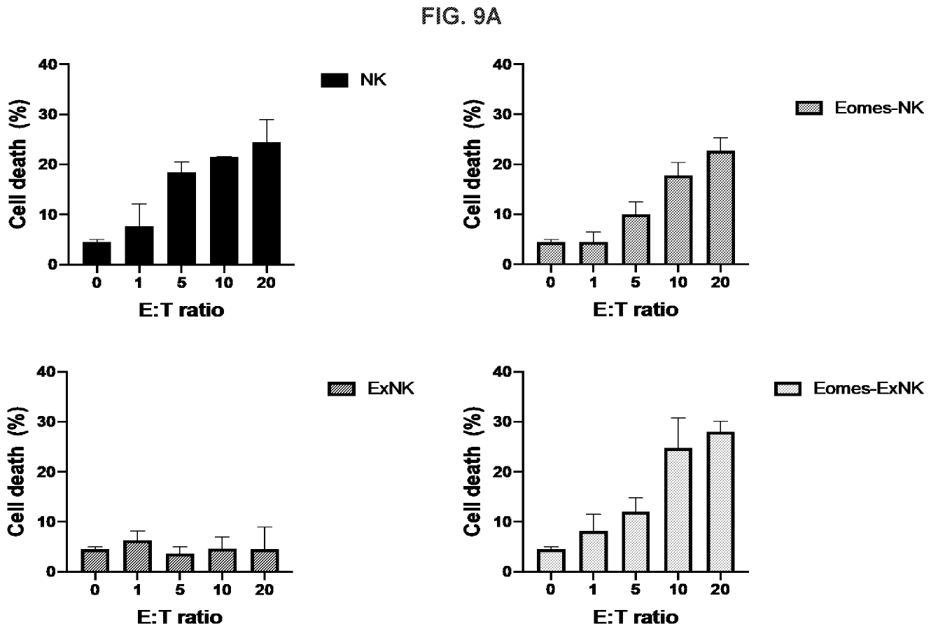

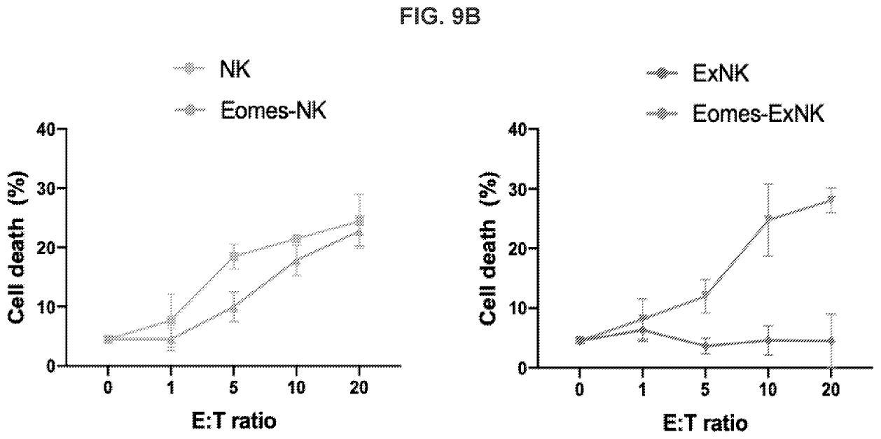

Cell death was analyzed using flow cytometry (FIG. 9). Experimental method of cell death was same as FIG. 8 experiment. The data showed that NK cells had increased cell death as the E:T ratio increased, while Eomes-NK cells had similar efficacy as NK cells. This indicated that Eomes did not have a significant effect on NK cells to increase their cytotoxicity.

On the other hand, ExNK and Eomes-ExNK showed distinctively different result. ExNK cells showed similar cell death amount as the E:T ratio was increased, because ExNK cells lost their cytotoxicity due to exhaustion. Interestingly, Eomes-ExNK cells recovered its cytotoxicity similar to NK cells. Eomes-ExNK cells had increased cell death amount as ET ratio was increased, and the cell death amount of Eomes-ExNK group was reached until that of NK group.

Collectively Eomes rejuvenated the cytotoxic reactivity of ExNK cells but not of NK cells. While ExNK cells did not have anti-cancer effect at every E:T ratio, Eomes-ExNK cells had increased anti-cancer effects like NK cells. In addition, the cytokine secretion level of Eomes-ExNK cells increased compared to ExNK cells. On the other hand, Eomes-NK cells had insignificant changes compared to NK cells, and they had similar effect of cytotoxicity and cytokine secretion with NK cells. These data demonstrated that Eomes could rejuvenate ExNK cells I but did not affect the anti-cancer activity of NK cells. Furthermore, Eomes-NK or -ExNK cells showed significantly higher GM-CSF amount than bare-NK or -ExNK cells. This result implied that rejuvenation mechanism of Eomes might be related to a GM-CSF signal pathway.

From the samples above, the cell supernatants were analyzed using cytokine ELISA. IFN-γ, TNF-α, Granzyme B, and GM-CSF were analyzed, because they are well-known cytokine secreted from NK cells. Cytokine release trend was similar with cancer cell death analysis. Eomes-NK cells did not have significant difference from NK cells, but Eomes-ExNK cells had increased cytokine release compared to ExNK cells. Also, the concentrations of cytokines from Eomes-ExNK cells were similar to that of NK cells. These cytokine ELISA result are similar to the results of the cancer cell death analysis. Interestingly, GM-CSF had different results than the other cytokines. GM-CSF concentration was increased in both Eomes-NK and Eomes-ExNK cells. This result could be related to downstream effects of Eomes on GM-CSF.

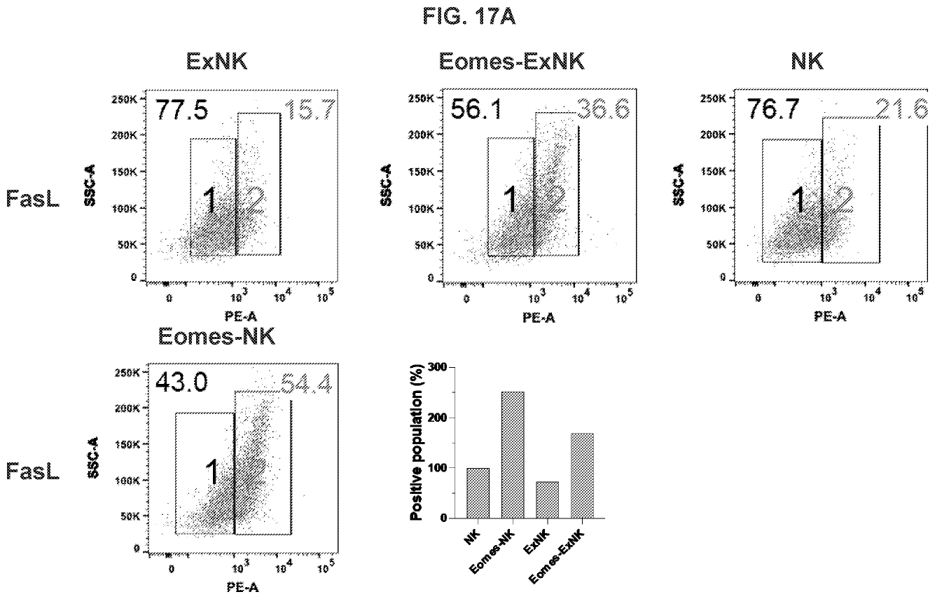

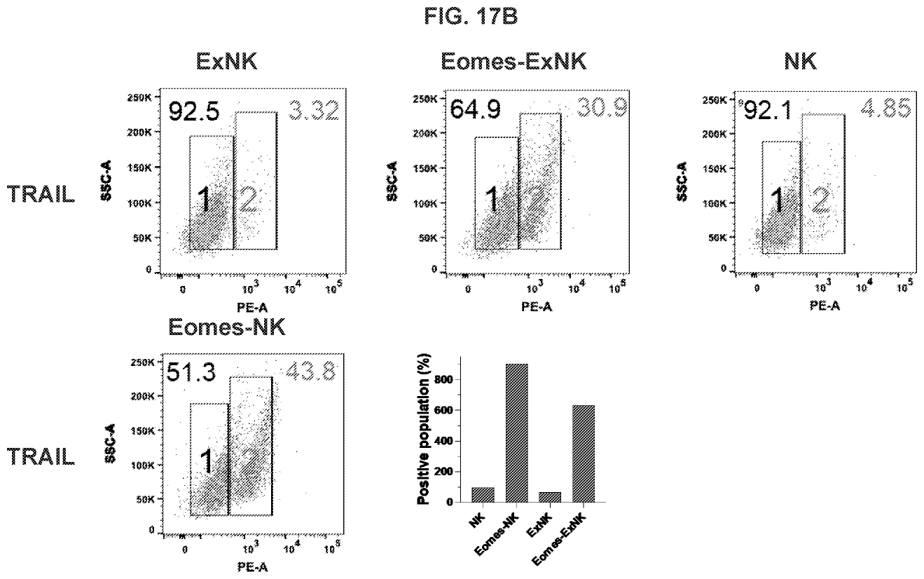

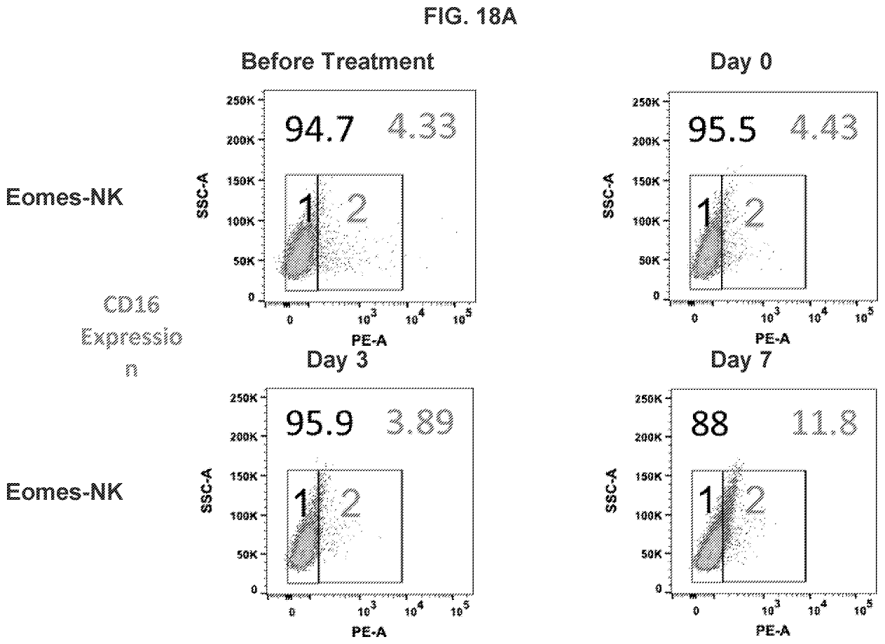

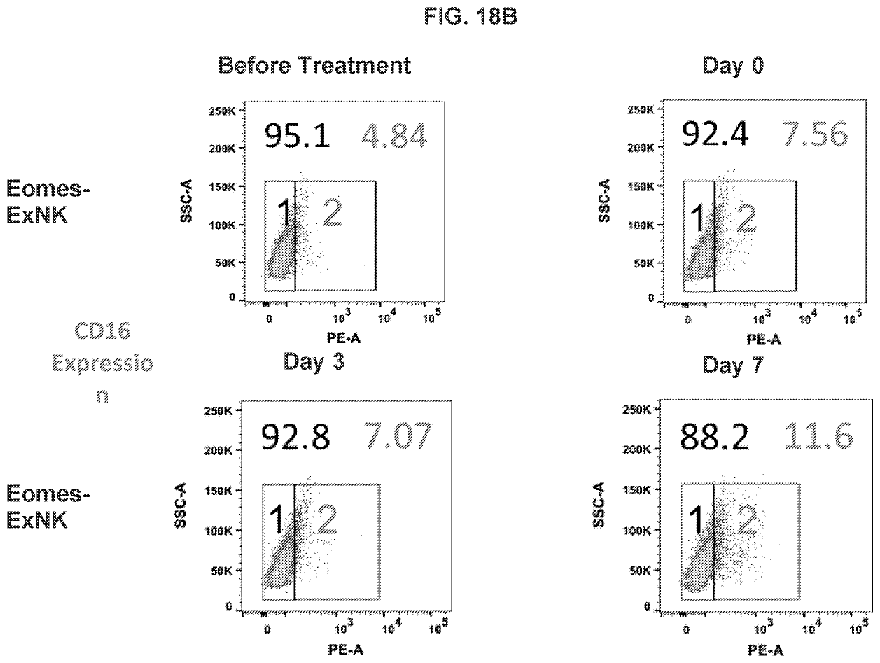

Additionally, Eomes induced increased expression of the death receptor ligands FasL and TRAIL for both NK and ExNK cells (FIG. 17A-B), which is in line with Eomes-mediated activated cytotoxicity. Furthermore, Eomes induced increased expression of the activation marker CD16 after 7 days post-transduction for both NK and ExNK cells (FIG. 18A-B), which is also in line with Eomes-mediated activated cytotoxicity.

### Eomes Effect on Anti-Cancer Activity of NK Cells Depending on Exhaustion State

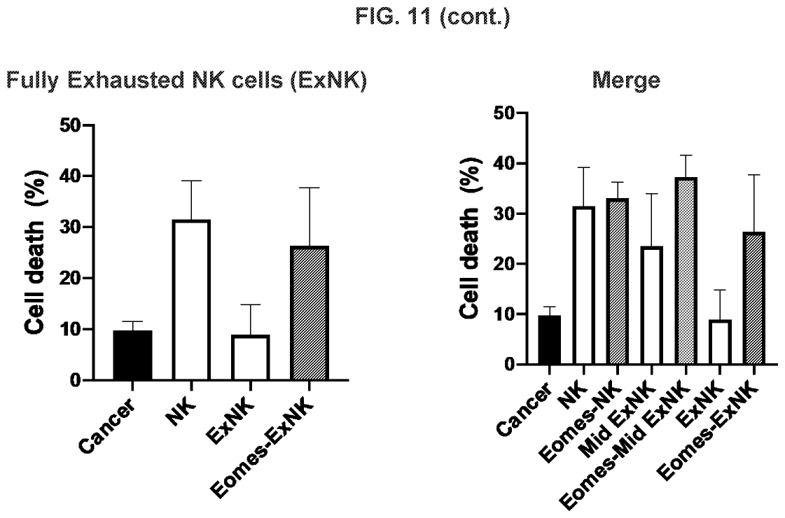

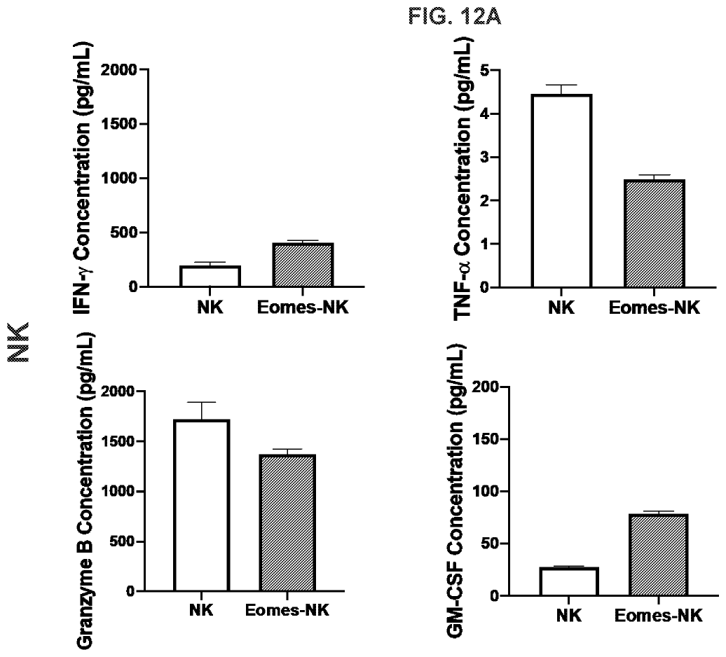

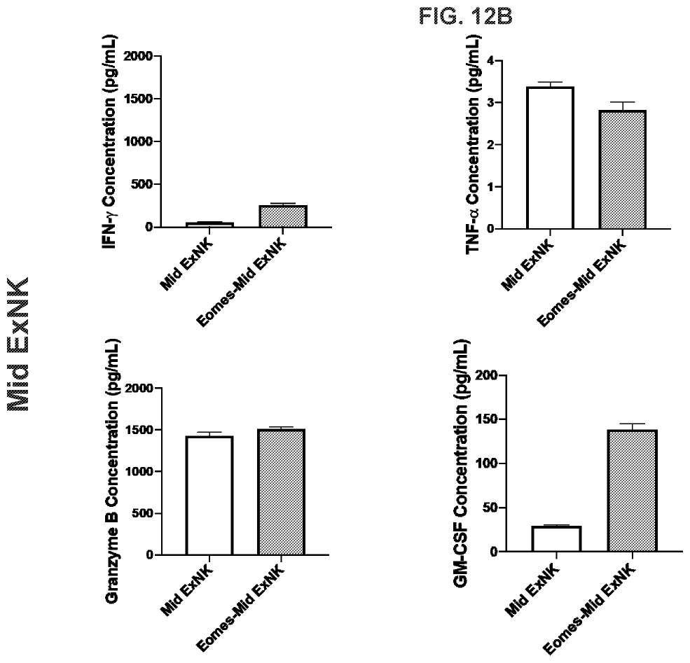

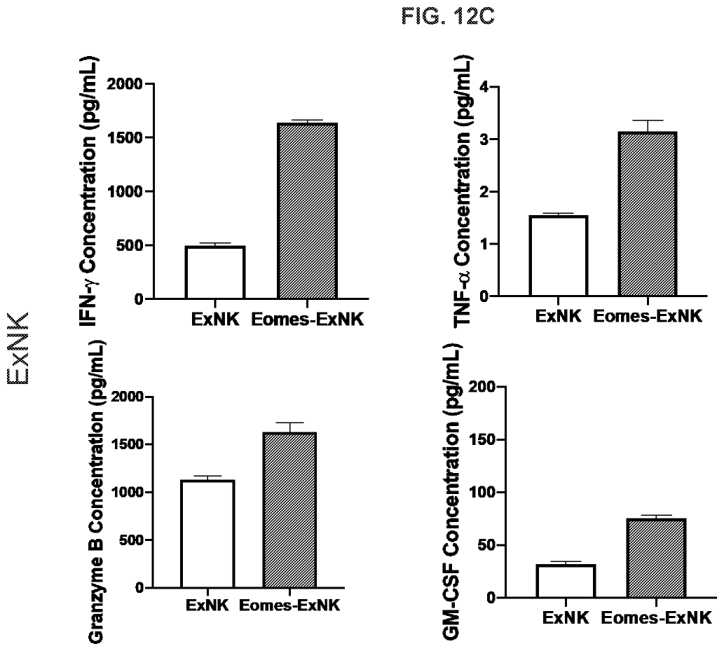

Different levels of exhausted NK cells including middle exhausted and (Mid ExNK) fully exhausted NK cells (ExNK) were prepared. Normal NK cells (NK), Mid ExNK, and ExNK cells were transduced with Eomes using the optimal transduction conditions. MDA-MB-231 cells were utilized as target cancer cells, and the E:T ratio was set at 5:1. Cells and supernatant media were separately collected and analyzed for cell death cytokine expression as described above. FIG. 11 shows that Eomes did not significantly increase cancer cell death with NK cells, but Eomes increased cancer cell death for both Mid ExNK and ExNK. Surprisingly, Eomes-Mid ExNK had caused greater cancer cell death than normal NK cells. This data indicated that Eomes could rejuvenate exhausted NK cells to normal NK cells regardless of exhaustion state.

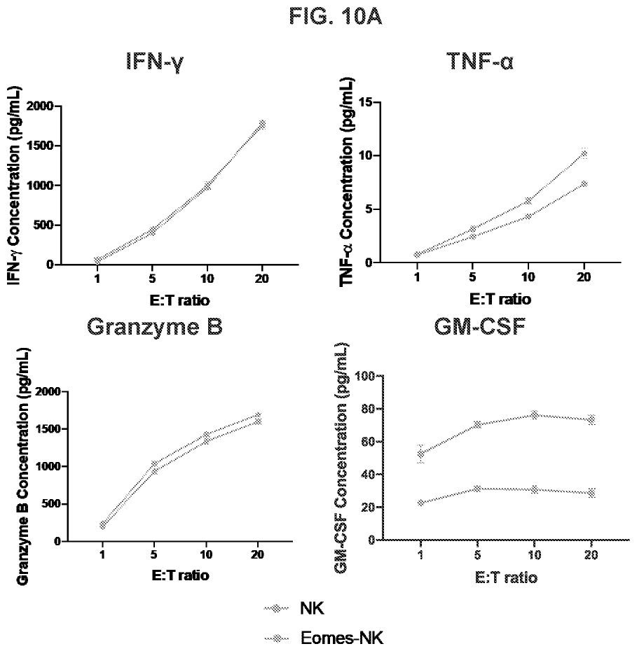

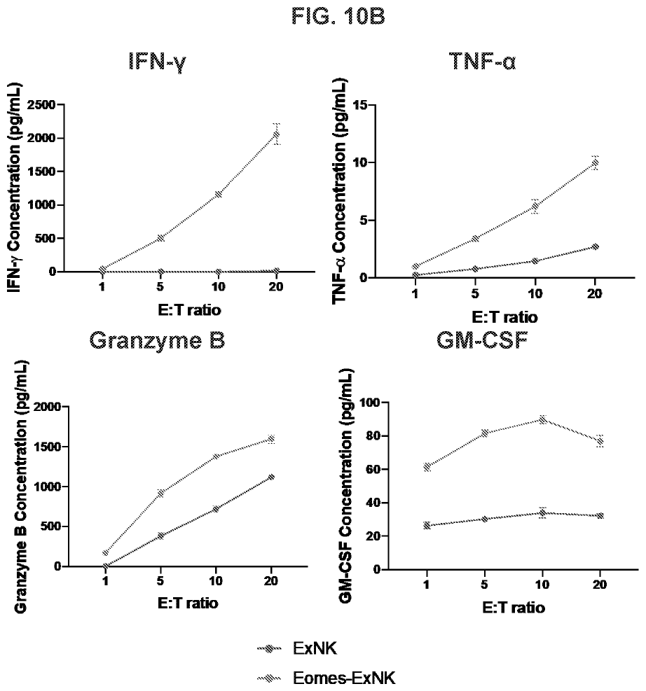

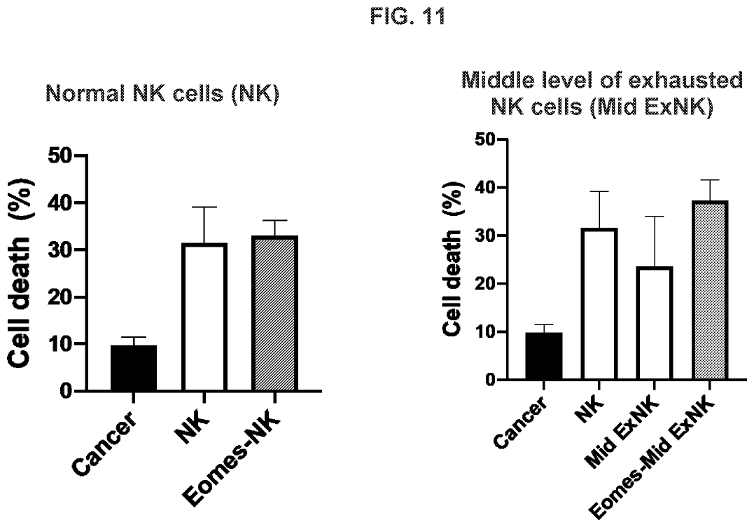

Supernatants from same samples were analyzed using cytokine ELISA. Eomes caused ExNK cells to secrete more cytokines compared to untreated ExNK cells. These data had similar trends as FIG. 10 because Eomes did not increase cytokine secretion on NK cells while it did on ExNK cells. Similar to FIG. 10, each NK type had increased GM-CSF secretion with Eomes transduction regardless of exhaustion stage. This result is in line with FIG. 11 that Eomes rejuvenates anti-cancer activity of ExNK cells.

### Eomes Effect on NK Cells' Inhibitory Marker Expression

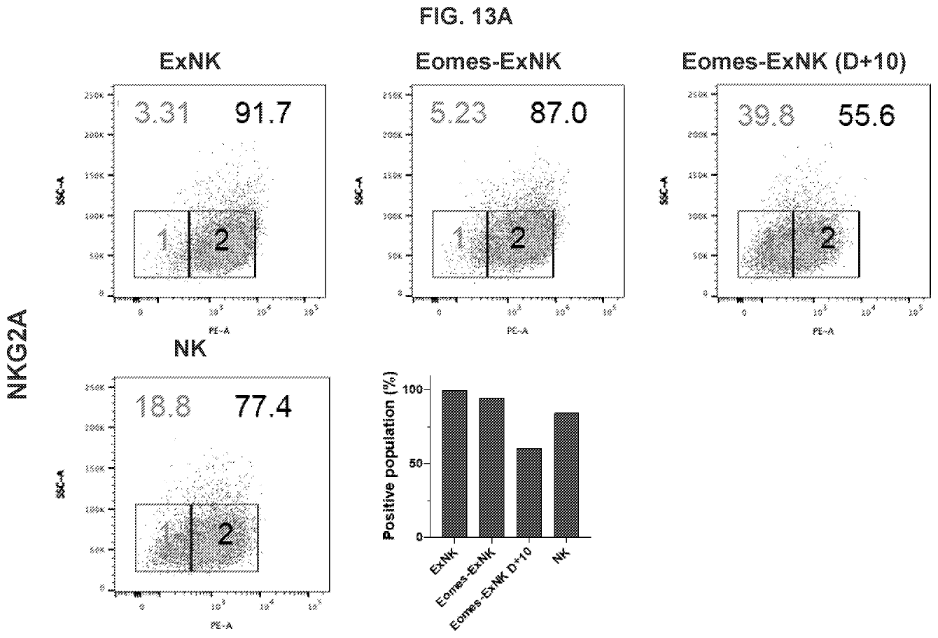

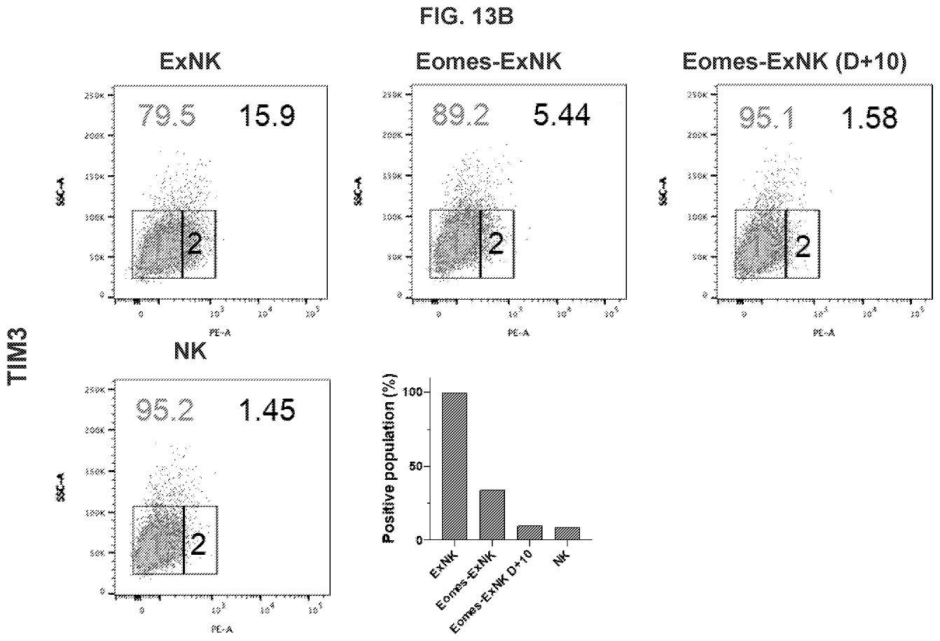

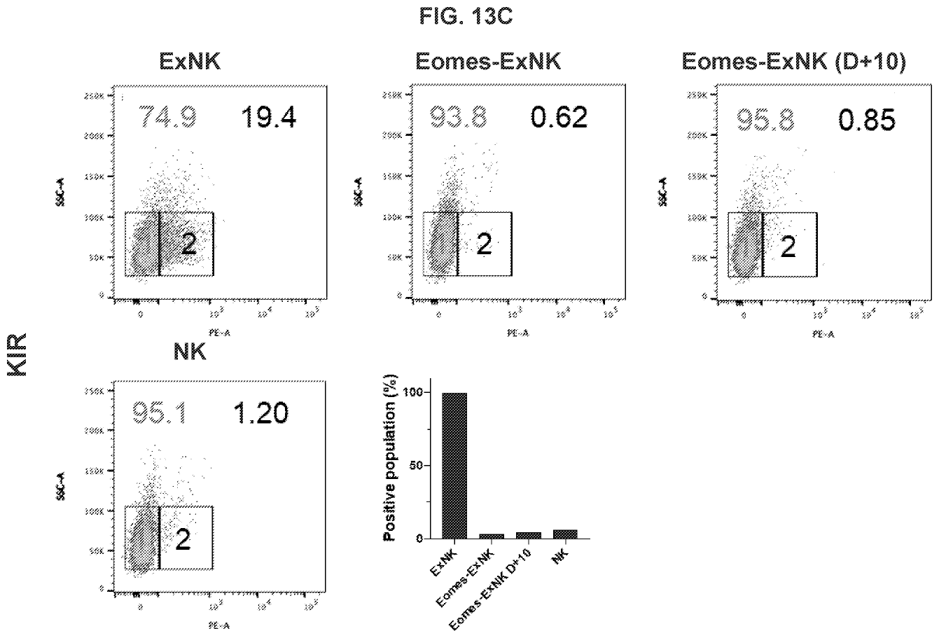

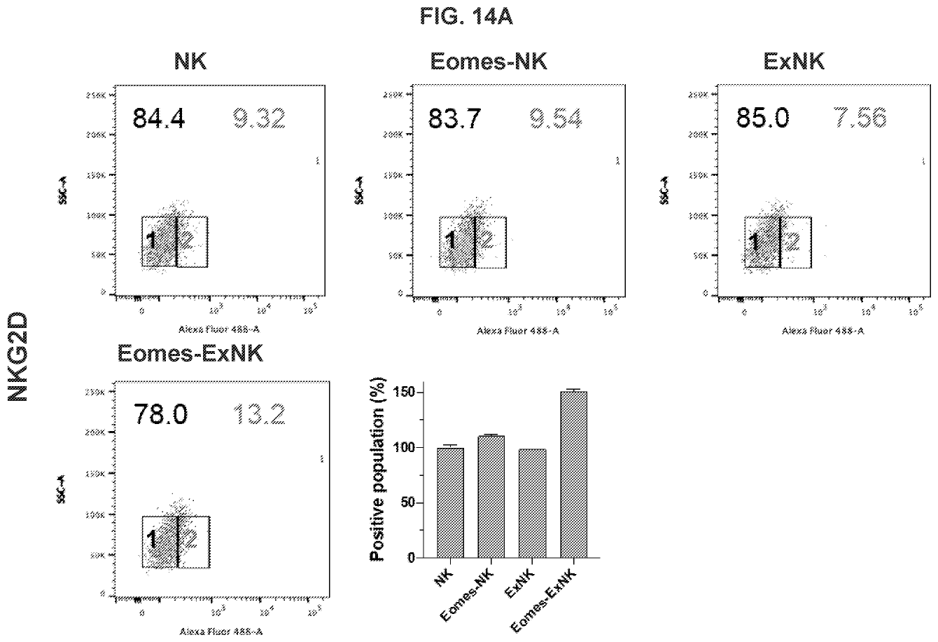

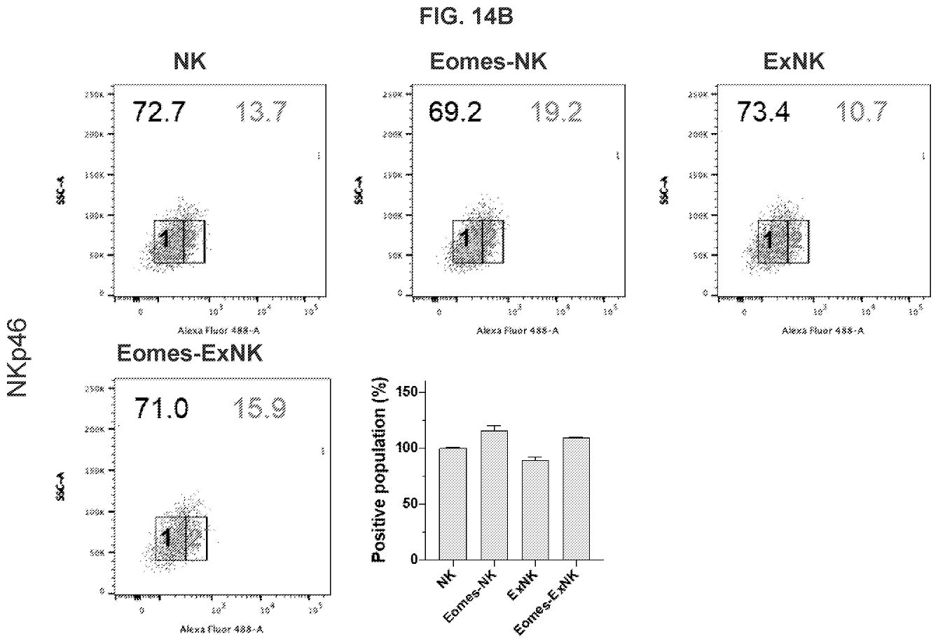

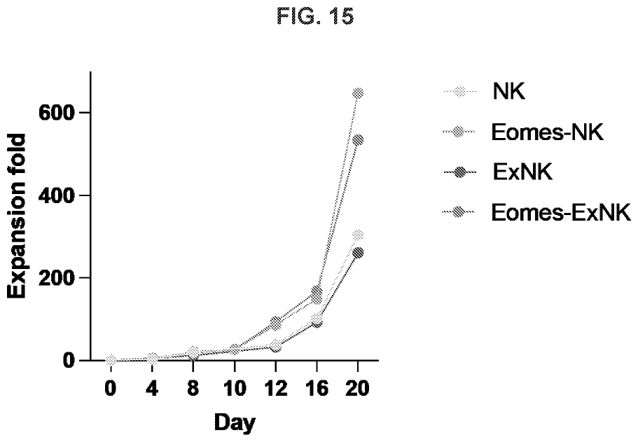

Eomes transduction was performed using the optimal transduction conditions. Eomes-ExNK cells and Eomes-ExNK cells D+10, were prepared and incubated for 10 days post-transduction. Normal NK, ExNK, Eomes-ExNK, and Eomes-ExNK D+10 cells were separately labeled with anti NKG2A, anti TIM3, anti KIR antibodies and these antibodies were conjugated with a PE fluorophore. The PE intensity of antibody-labeled cells was measured using flow cytometry (BD LSRII) (FIG. 13). In the dot plots of FIG. 13, red and black colored numbers represent number 1 and 2 boxes, respectively. Number 1 box is the cell population of low PE intensity, and it indicates low expression of inhibitory markers. The number 2 box represents high expression of inhibitory markers. As the black colored numbers show in FIG. 13, ExNK cells had high expression level of inhibitory markers compared to NK cells, and expression level of inhibitory markers of both Eomes-ExNK and Eomes-ExNK D+10 cells reached that of NK cells. Furthermore, Eomes-ExNK D+10 cells had decreased expression levels of inhibitory marker that were in line with that Eomes-mediated rejuvenation effect could persist until 10 days. These results demonstrated that Eomes supressed expression of inhibitory marker of ExNK cells, rejuvenated the ExNK cells, and the effect could last for 10 days with a single transduction.

Eomes-ExNK showed decreased expression of inhibitory markers compared to untreated ExNK cells. The marker expression of Eomes-ExNK was decreased to the level of normal NK cells, implying that Eomes-ExNK were rejuvenated to levels of normal NK cells. The decreased expressions of inhibitory markers were maintained for 10 days post-transduction.

### Example 4

### In Vivo Efficacy Study Procedure

### Preparation of NK, Exhausted NK (ExNK), Eomes-NK, and Eomes-ExNK

Various Eomes concentration and incubation time for transduction were validated on NK and ExNK cells for optimal transduction condition. 0.5 μM Eomes and 3 h transduction time were optimized to transduce Eomes on 1×10⁶ NK and ExNK cells (Eomes-NK and Eomes-ExNK).

### MDA-MB-231 Model Generation and Injection

Female NSG mice (NOD.Cg-Prkdcscid II2rgtm1Wjl/SzJ) were inoculated with 1×10⁶ MDA-MB-231 cells (triple negative breast cancer cells) suspended in 50% Matrigel. The number of mice were equally divided for control (non-treatment), NK, ExNK, Eomes-NK, and Eomes-ExNK groups. Once the tumor size reached about 150 mm³, weekly injection of NK, ExNK, NK-Eomes, and ExNK-Eomes was commenced Every cell group was injected at a dosage of 1×10⁷ cells in 100 μL of HBSS. Animals were intravenously injected with the respective treatment for 4 weeks. All animal procedures followed the guidelines approved by the University of Arizona IACUC.

### Tumor Volume and Mouse Body Weight Measurement

Tumor volume and mouse body weight were recorded every once a week. Tumor volume was obtained by measurements of the length and the width of the tumor with a caliper and calculated using the formula: V=0.5·ab², where (a) is the longest diameter and (b) is the shortest diameter of the tumor.

### Tumor Tissue Excision

After 4 weeks of injection is done, mice were euthanized, and tumor tissue was harvested. The tissues were stored at −80° C. for further analysis, such as flow cytometry or cytokine ELISA.

### In Vivo Anti-Cancer Efficacy Study Results

### Tumor Growth Suppression

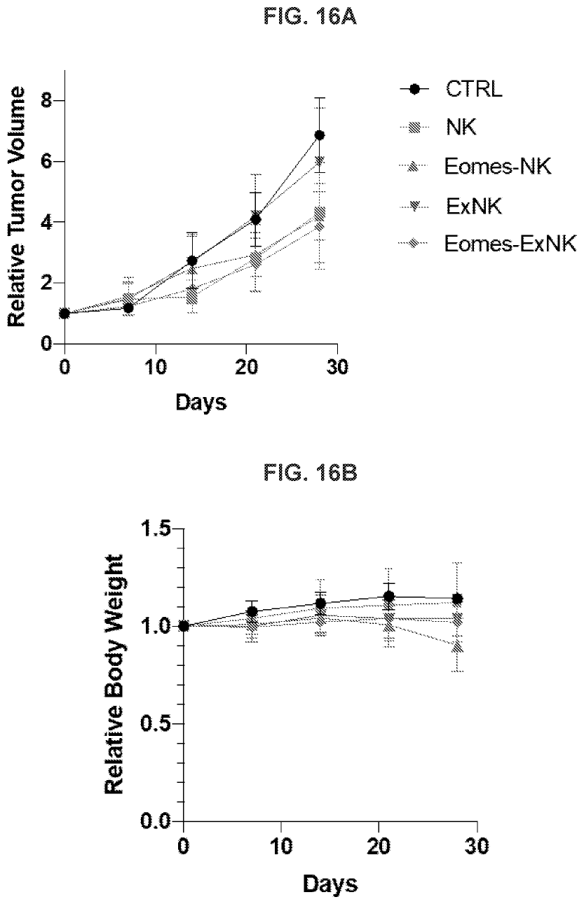

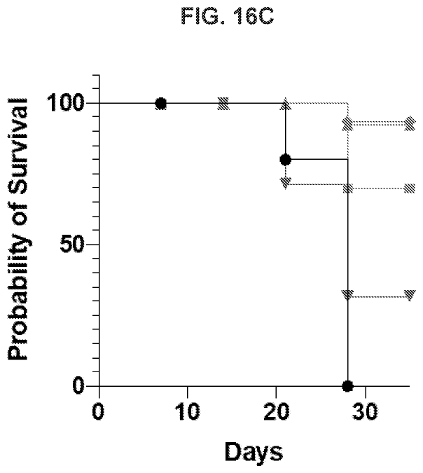

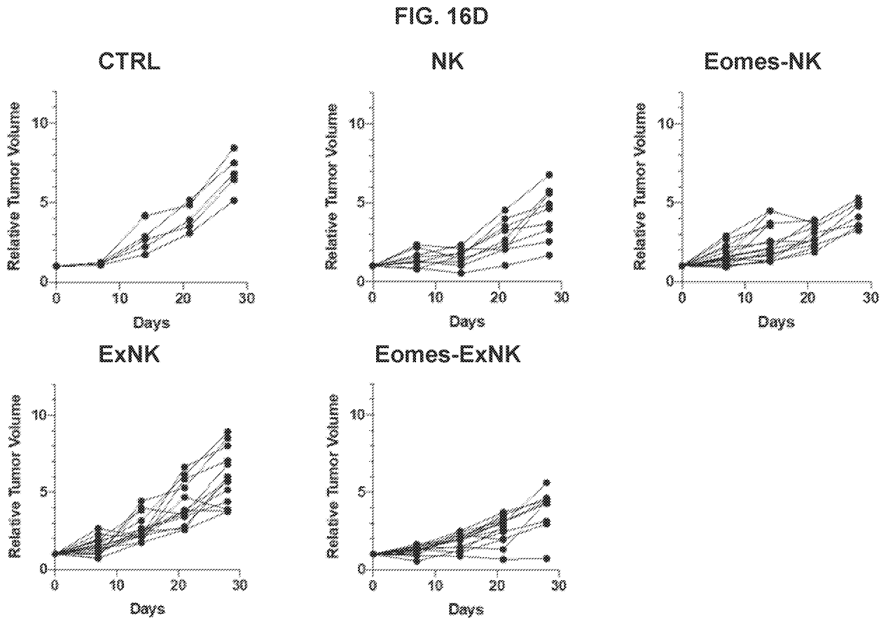

Tumor growth was monitored as control (non-treatment), NK, Eomes-NK, ExNK, Eomes-ExNK groups. The Eomes-ExNK group showed similar inhibition on tumor growth as the NK group, while ExNK did not show tumor growth inhibition (FIG. 16A-D). The Eomes-ExNK group showed improved tumor growth inhibition compared to ExNK. These cells inhibited tumor growth at similar levels to the NK. Unlike ExNK cells, Eomes-NK cells did not have a significant difference from NK cells. This observed anti-cancer effect is in line with the in vitro assays (FIGS. 9 and 11). These data verified that Eomes recovered anti-cancer activity of ExNK cells. Additional in vivo experiments are ongoing. These results demonstrated that Eomes-ExNK has promising applications on cancer therapy by rejuvenating ExNK cells. The Eomes could be utilized for autologous NK cell injection of immune-defected patients.

What is claimed:

1. A nucleotide sequence encoding a polypeptide, wherein the polypeptide comprises an Eomesodermin domain, and one or more nuclear localization signals and/or protein-transduction domain sequences.

2. The nucleotide sequence of claim 1, wherein the Eomesodermin domain comprises an Eomesodermin transcription factor associated domain.

3. The nucleotide sequence of claim 1 or 2, wherein the polypeptide comprises one or more nuclear localization signals, the Eomesodermin transcription factor associated domain, and one or more protein-transduction domains, each optionally separated by a glycine-serine linker.

4. The nucleotide sequence of claims 1-3, wherein the polypeptide has the structure:
NLS-GL-Eomes-TFAD-GL-PTD;
PTD-GL-Eomes-TFAD-GL-NLS;
PTD-GL-Eomes-TFAD; or
NLS-GL-Eomes-TFAD;
wherein NLS is a nuclear localization signal; GL is an optional glycine-serine linker;
Eomes-TAD is an Eomesodermin transcription factor associated domain, PTD is a protein-transduction domain.

5. The nucleotide sequence of claims 1-4, wherein the polypeptide further comprises an affinity tag.

6. The nucleotide sequence of claims 1-5, wherein the affinity tag comprises a 6×-histidine tag.

7. The nucleotide sequence of claims 1-6, wherein the Eomesodermin transcription factor associated domain comprises SEQ ID NO: 2 or 4.

8. The nucleotide sequence of claims 1-7, wherein the Eomesodermin transcription factor associated domain comprises residues 267-686 of SEQ ID NO: 2 or 4.

9. The nucleotide sequence of claims 1-8, wherein the nuclear localization signal comprises one or more of SEQ ID NO: 7-11 and the protein-transduction domain comprises one or more of SEQ ID NO: 13-27.

10. The nucleotide sequence of claims 1-9, wherein the nuclear localization signal comprises residues 16-21 of SEQ ID NO: 7; the protein-transduction domain comprises residues 1-11 of SEQ ID NO: 13; and the glycine-serine linker comprises SEQ ID NO: 28.

11. The nucleotide sequence of claims 1-10, wherein the nucleotide sequence has 85% to 99% identity to SEQ ID NO: 1.

12. The nucleotide sequence of claims 1-11, wherein the nucleotide sequence is SEQ ID NO: 1.

13. A polynucleotide vector comprising a nucleotide sequence of claims 1-12.

14. A cell comprising one or more nucleotide sequences of claim 1-12 or a polynucleotide vector of claim 13.

15. A polypeptide encoded by the nucleotide sequence of claims 1-12.

16. A polypeptide encoded by the nucleotide sequence of claims 1-12, wherein the polypeptide has 85% to 99% identity to SEQ ID NO: 2.

17. A polypeptide encoded by the nucleotide sequence of claims 1-12, wherein the polypeptide is SEQ ID NO: 2.

18. A research tool comprising a polypeptide encoded by the nucleotide sequence of claims 1-12.

19. A therapeutic reagent comprising a polypeptide encoded by the nucleotide sequence of claims 1-12.

20. A means or method for manufacturing the nucleotide sequence of claims 1-12 or a polypeptide encoded by the nucleotide sequence of claims 1-12, the process comprising: transforming or transfecting a cell with a nucleic acid comprising the nucleotide sequence of claim 1; growing the cells; optionally, harvesting the cells and isolating quantities of the nucleotide sequence of claims 1-12; inducing expression of a polypeptide encoded by the nucleotide sequence of claim 1-12; harvesting the cells; and isolating and purifying the polypeptide.

21. The nucleotide sequence of claims 1-12 or a polypeptide encoded by the nucleotide sequence of claims 1-12, each produced by the means or method of claim 20.

22. A therapeutic polypeptide comprising the amino acid sequence of SEQ ID NO: 2.

23. A method for rejuvenating exhausted natural killer cells, the method comprising:
obtaining exhausted natural killer cells (ExNK);
contacting the natural killer cells with a quantity of a polypeptide comprising one or more nuclear localization signals, an Eomesodermin transcription factor associated domain, and one or more protein-transduction domains;
incubating the ExNK and polypeptide for a period of time sufficient for the polypeptide to be uptaken by the cells (transduced); and
allowing the cells to incubate for a period of time sufficient for the polypeptide to rejuvenate the ExNKs.

24. A method for treating cancer, the method comprising:
(a) administering a therapeutically effective amount of polypeptide comprising one or more nuclear localization signals, an Eomesodermin transcription factor associated domain, and one or more protein-transduction domains to a subject in need thereof; or
(b) contacting autologous exhausted natural killer cells isolated from a subject with a therapeutically effective amount of polypeptide comprising one or more nuclear localization signals, an Eomesodermin transcription factor associated domain, and one or more protein-transduction domains; permitting the cells to rejuvenate; and administering the cells to a subject in need thereof.

25. Use of a polypeptide comprising one or more nuclear localization signals, an Eomesodermin transcription factor associated domain, and one or more protein-transduction domains for the rejuvenation of exhausted natural killer cells or the treatment of a subject in need thereof.
